(19) United States
(12) Patent Application Publication
(10) Pub. No.: US 2017/0007663 A1
(43) Pub. Date: Jan. 12, 2017

(54) METHODS AND COMPOSITIONS FOR TREATING AND PREVENTING COGNITIVE DYSFUNCTION

(71) Applicant: CORNELL UNIVERSITY, Ithaca, NY (US)

(72) Inventors: Hazel Szeto, New York, NY (US); Nicholas Schiff, New York, NY (US)

(21) Appl. No.: 15/104,849

(22) PCT Filed: Dec. 15, 2014
(86) PCT No.: PCT/US2014/070398

§ 371 Date: Jun. 15, 2016

Related U.S. Application Data
(60) Provisional application No. 61/916,647, filed on Dec. 16, 2013.

(51) Int. Cl. A61K 38/07

(52) U.S. Cl. CPC A61K 38/07

(57) ABSTRACT

The disclosure relates to methods and compositions for preventing or treating cognitive dysfunction. The methods comprise administering an effective amount of an aromatic-cationic peptide to subjects in need thereof. Specifically, the aromatic-cationic peptide has the formula D-Arg-2′,6′-Dmt-Lys-Phe-NH2, and the cognitive dysfunction is associated with a reduced cerebral metabolic rate of oxygen (CMRO).

### CROSS-REFERENCE TO RELATED APPLICATIONS

This application claims the benefit of and priority to U.S. Application No. 61/916,647 filed on Dec. 16, 2013, the content of which is incorporated by reference herein in its entirety.

### GOVERNMENT SUPPORT

This invention was made with government support under NS067249 awarded by the National Institute of Health. The government has certain rights in the invention.

### TECHNICAL FIELD

The present technology relates generally to compositions and methods for enhancing cerebral function. In particular, the present technology relates to methods and compositions that improve cognitive function and behavioral response in diseases or conditions where neuronal function has been compromised or degraded.

### BACKGROUND

The following description is provided to assist the understanding of the reader. None of the information provided or references cited is admitted to be prior art to the present technology.

Cognitive dysfunction is the loss of intellectual functions such as thinking, remembering, and reasoning of sufficient severity to interfere with daily functioning. Persons or patients with cognitive dysfunction have trouble, for example, with verbal recall, basic arithmetic, and concentration.

Cognitive dysfunction can be associated with deafferentation. The cause of deafferentation can be intrinsic (e.g., a condition or disease a subject was born with) or acquired (e.g., brain trauma or head injury after birth). Cognitive dysfunction can also be associated with a reduced cerebral metabolic rate of oxygen (CMRO). Similar to deafferentation, CMRO can be instigated by intrinsic or extrinsic factors.

### SUMMARY

The present technology relates generally to the treatment and prevention of cognitive dysfunction through administration of therapeutically effective amounts of aromatic-cationic peptides to subjects in need thereof. In some embodiments, the cognitive dysfunction is associated with a decrease in cerebral metabolic rate of oxygen (CMRO)

In one aspect, the present disclosure provides a method to treat and/or prevent cognitive dysfunction in a mammalian subject in need thereof, the method comprising administering to the subject a therapeutically effective amount of the peptide D-Arg-2′,6′-dimethyltyrosine-Lys-Phe-NH₂ (dimethyltyrosine is represent by Dmt).

In one aspect, the disclosure provides a method of treating or preventing cognitive dysfunction in a mammalian subject, comprising administering to said mammalian subject a therapeutically effective amount of an aromatic-cationic peptide. In some embodiments, the aromatic-cationic peptide is a peptide having:

at least one net positive charge;

a minimum of four amino acids;

a maximum of about twenty amino acids;

a relationship between the minimum number of net positive charges (pₘ) and the total number of amino acid residues (r) wherein 3pₘ is the largest number that is less than or equal to r+1; and a relationship between the minimum number of aromatic groups (a) and the total number of net positive charges (pₜ) wherein 2a is the largest number that is less than or equal to pₜ+1, except that when a is 1, pₜ may also be 1. In particular embodiments, the mammalian subject is a human.

In one embodiment, 2pₘ is the largest number that is less than or equal to r+1, and may be equal to pₜ. The aromatic-cationic peptide may be a water-soluble peptide having a minimum of two or a minimum of three positive charges.

In one embodiment, the peptide comprises one or more non-naturally occurring amino acids, for example, one or more D-amino acids. In some embodiments, the C-terminal carboxyl group of the amino acid at the C-terminus is amidated. In certain embodiments, the peptide has a minimum of four amino acids. The peptide may have a maximum of about 6, a maximum of about 9, or a maximum of about 12 amino acids.

In one embodiment, the peptide may have the formula Phe-D-Arg-Phe-Lys-NH₂ or 2′,6′-Dmp-D-Arg-Phe-Lys-NH₂ (Dmp refers to dimethylphenylalanine). In a particular embodiment, the aromatic-cationic peptide has the formula D-Arg-2′,6′-Dmt-Lys-Phe-NH₂.

In one embodiment, the peptide is defined by formula I:

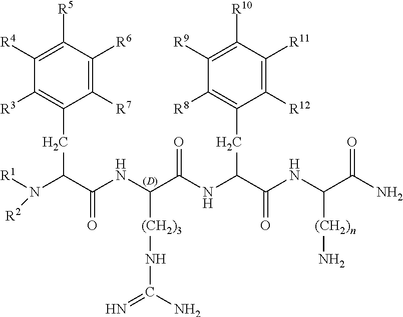

wherein R¹ and R² are each independently selected from

(i) hydrogen;

(ii) linear or branched C₁-C₆ alkyl;

(iii)

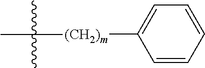

where m=1-3;

(iv)

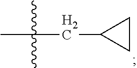 [SMILES: *CC1CC1]

(v)

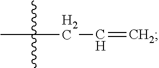 [SMILES: *CC=C]

R³, R⁴, R⁵, R⁶, R⁷, R⁸, R⁹, R¹⁰, R¹¹ and R¹² are each independently selected from

(i) hydrogen;

(ii) linear or branched C₁-C₆ alkyl;

(iii) C₁-C₆ alkoxy;

(iv) amino;

(v) C₁-C₄ alkylamino;

(vi) C₁-C₄ dialkylamino;

(vii) nitro;

(viii) hydroxyl;

(ix) halogen, where “halogen” encompasses chloro, fluoro, bromo, and iodo; and n is an integer from 1 to 5.

In a particular embodiment, R¹, R², R³, R⁴, R⁵, R⁶, R⁷, R⁸, R⁹, R¹⁰, R¹¹, and R¹² are all hydrogen; and n is 4. In another embodiment, R¹, R², R³, R⁴, R⁵, R⁶, R⁷, R⁸, R⁹, and R¹¹ are all hydrogen; R⁸ and R¹² are methyl; R¹⁰ is hydroxyl; and n is 4.

In one embodiment, the peptide is defined by formula II:

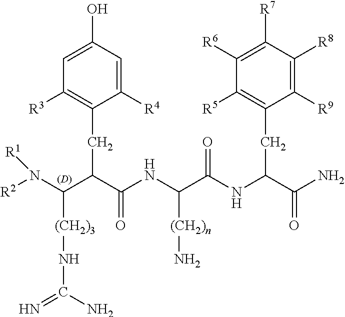

wherein R¹ and R² are each independently selected from

(i) hydrogen;

(ii) linear or branched C₁-C₆ alkyl;

(iii)

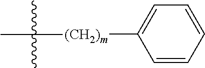

when m=1-3;

(iv)

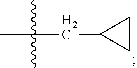 [SMILES: *CC1CC1]

(v)

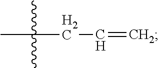 [SMILES: *CC=C]

R³ and R⁴ are each independently selected from

(i) hydrogen;

(ii) linear or branched C₁-C₆ alkyl;

(iii) C₁-C₆ alkoxy;

(iv) amino;

(v) C₁-C₄ alkylamino;

(vi) C₁-C₄ dialkylamino;

(vii) nitro;

(viii) hydroxyl;

(ix) halogen, where “halogen” encompasses chloro, fluoro, bromo, and iodo;

R⁵, R⁶, R⁷, R⁸, and R⁹ are each independently selected from

(i) hydrogen;

(ii) linear or branched C₁-C₆ alkyl;

(iii) C₁-C₆ alkoxy;

(iv) amino;

(v) C₁-C₄ alkylamino;

(vi) C₁-C₄ dialkylamino;

(vii) nitro;

(viii) hydroxyl;

(ix) halogen, where “halogen” encompasses chloro, fluoro, bromo, and iodo; and n is an integer from 1 to 5.

In some embodiments, the subject is a human.

In some embodiments, the peptide is administered intraocularly, iontophoretically, orally, topically, systemically, intravenously, subcutaneously, or intramuscularly.

In some embodiments, the cognitive dysfunction is related to or caused by a genetic mitochondrial disorder. Additionally or alternatively, in some embodiments, the cognitive dysfunction is related to decreased cerebral metabolic oxygen rate. Additionally or alternatively, in some embodiments, the cognitive dysfunction is related to deafferentation. Additionally or alternatively, in some embodiments, the cognitive dysfunction is related to non-progressive brain injury.

In another aspect, the present technology provides methods for treating or preventing post-operative cognitive dysfunction, (e.g., anesthesia-induced cognitive dysfunction) in a mammalian subject in need thereof, comprising administering to the subject a therapeutically effective amount of a peptide represented by the formula D-Arg-2′,6′-Dmt-Lys-Phe-NH₂ or a pharmaceutically acceptable salt thereof.

In another aspect, the present technology provides methods for treating or preventing cognitive dysfunction caused by drug-induced mitochondrial side effects in a mammalian subject in need thereof, comprising administering to the subject a therapeutically effective amount of a peptide represented by the formula D-Arg-2′,6′-Dmt-Lys-Phe-NH₂ or a pharmaceutically acceptable salt thereof.

In another aspect, the present technology provides methods for treating or preventing cognitive dysfunction in a subject diagnosed with or at increased risk of Lewy Body Disease, comprising administering to the subject a therapeutically effective amount of a peptide represented by the formula D-Arg-2′,6′-Dmt-Lys-Phe-NH₂ or a pharmaceutically acceptable salt thereof.

In some embodiments, any of the above methods further comprises administering at least one second therapeutic active agent.

### BRIEF DESCRIPTION OF DRAWINGS

FIGS. 1A and 1B are schematics adapted from Ching et al, PNAS, 109(8): 3095-3100 (Feb. 21, 2012). FIG. 1A is a schematic showing reduced neuronal electrical activity in neuronal cellular and network function as measured by electroencephalography (EEG) in a setting of functional or structural deafferentation. FIG. 1B is a schematic showing restoration of electrical activity in the neuronal cellular and network function of FIG. 1A after treatment with at least one aromatic-cationic peptide, e.g., D-Arg-2′,6′-Dmt-Lys-Phe-NH₂.

### DETAILED DESCRIPTION

It is to be appreciated that certain aspects, modes, embodiments, variations and features of the invention are described below in various levels of detail in order to provide a substantial understanding of the present invention.

In practicing the present invention, many conventional techniques in molecular biology, protein biochemistry, cell biology, immunology, microbiology and recombinant DNA are used. These techniques are well-known and are explained in, e.g., Current Protocols in Molecular Biology, Vols. I-III, Ausubel, Ed. (1997); Sambrook et al., Molecular Cloning: A Laboratory Manual, Second Ed. (Cold Spring Harbor Laboratory Press, Cold Spring Harbor, N.Y., 1989); DNA Cloning: A Practical Approach, Vols. I and II, Glover, Ed. (1985); Oligonucleotide Synthesis, Gait, Ed. (1984); Nucleic Acid Hybridization, Hames & Higgins, Eds. (1985); Transcription and Translation, Hames & Higgins, Eds. (1984); Animal Cell Culture, Freshney, Ed. (1986); Immobilized Cells and Enzymes (IRL Press, 1986); Perbal, A Practical Guide to Molecular Cloning; the series, Meth. Enzymol., (Academic Press. Inc., 1984); Gene Transfer Vectors for Mammalian Cells, Miller & Calos, Eds. (Cold Spring Harbor Laboratory, N Y, 1987); and Meth. Enzymol., Vols. 154 and 155, Wu & Grossman, and Wu, Eds., respectively.

The definitions of certain terms as used in this specification are provided below. Unless defined otherwise, all technical and scientific terms used herein generally have the same meaning as commonly understood by one of ordinary skill in the art to which this invention belongs.

As used in this specification and the appended claims, the singular forms “a”, “an” and “the” include plural referents unless the content clearly dictates otherwise. For example, reference to “a cell” includes a combination of two or more cells, and the like.

As used herein, “about” will be understood by persons of ordinary skill in the art and will vary to some extent depending upon the context in which it is used. If there are uses of the term which are not clear to persons of ordinary skill in the art, given the context in which it is used, “about” will mean up to plus or minus 10% of the enumerated value.

As used herein, the “administration” of an agent, drug, or peptide to a subject includes any route of introducing or delivering to a subject a compound to perform its intended function. Administration can be carried out by any suitable route, including orally, intraocularly, intranasally, parenterally (intravenously, intramuscularly, intraperitoneally, or subcutaneously), or topically. Administration includes self-administration and the administration by another.

As used herein, the term “amino acid” includes naturally-occurring amino acids and synthetic amino acids, as well as amino acid analogs and amino acid mimetics that function in a manner similar to the naturally-occurring amino acids. Naturally-occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally-occurring amino acid, i.e., an α-carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally-occurring amino acid. Amino acid mimetics refers to chemical compounds that have a structure that is different from the general chemical structure of an amino acid, but that functions in a manner similar to a naturally-occurring amino acid. Amino acids can be referred to herein by either their commonly known three letter symbols or by the one-letter symbols recommended by the IUPAC-IUB Biochemical Nomenclature Commission.

As used herein, the term “cognitive dysfunction” refers to an impaired ability to perform high-level brain functions, which include but are not limited to, the ability to learn and remember information, organize, plan, problem-solve, focus, maintain and shift attention as necessary, understand and use language, accurately perceive the environment, and perform calculations. In some embodiments, cognitive dysfunction is associated with deafferentation. In some embodiments, the cognitive dysfunction is associated with a reduction in cerebral metabolic rate of oxygen (CMRO). By way of example, but not by way of limitation, subjects with deafferentation and/or reduced CMRO include, but are not limited to, subjects undergoing or recovering from anesthesia, subjects that suffered a non-progressive brain injury, subjects suffering from drug-induced mitochondrial side effects, subjects diagnosed with a mitochondrial genetic disease or disorder, and subjects diagnosed with Lewy Body Disease.

As used herein, the term “effective amount” refers to a quantity sufficient to achieve a desired therapeutic and/or prophylactic effect, e.g., an amount which results in the increase of cerebral metabolic rate of oxygen and/or supports activation of neuronal elements within the brain leading to improved cognitive function and behavioral responsiveness. The amount of a composition administered to the subject will depend on the type and severity of the disease and on the characteristics of the individual, such as general health, age, sex, body weight and tolerance to drugs. It will also depend on the degree, severity and type of disease. The skilled artisan will be able to determine appropriate dosages depending on these and other factors. The compositions can also be administered in combination with one or more additional therapeutic compounds. In the methods described herein, the aromatic-cationic peptides may be administered to a subject having one or more signs or symptoms of altered or decreased neuronal activity and/or deafferentation of neurons. By way of example, but not by way of limitation, a “therapeutically effective amount” of the aromatic-cationic peptides is levels in which the neurological or physiological effects of a neurodegenerative disease, such as mitochondrial encephalopathies are, at a minimum, ameliorated.

As used herein, the term “deafferentation” refers to the elimination, reduction, or interruption of sensory nerve impulses. In some embodiments, the elimination, reduction, or interruption of sensory nerve impulses leads to a reduction in cerebral metabolic rate of oxygen (CMRO). In some embodiments, deafferentation is due to acquired dysfunction and/or an intrinsic dysfunction. By way of example, but not by limitation, in some embodiments, cognitive dysfunction results from an intrinsic disorder, e.g., a mitochondrial genetic disorder, e.g., by way of example, but not by way of limitation, mitochondrial encephalomyopathy, or an acquired disorder, e.g., by way of example, but not by way of limitation, anesthetic application, brain injury, etc.

As used herein, the term “subject” and “patient” are used interchangeably and include mammals, for example, farm animals, such as sheep, pigs, cows, and horses; pet animals, such as dogs and cats; laboratory animals, such as rats, mice and rabbits. In some embodiments, the mammal is a human.

An “isolated” or “purified” polypeptide or peptide is substantially free of cellular material or other contaminating polypeptides from the cell or tissue source from which the agent is derived, or substantially free from chemical precursors or other chemicals when chemically synthesized. For example, an isolated aromatic-cationic peptide would be free of materials that would interfere with diagnostic or therapeutic uses of the agent. Such interfering materials may include enzymes, hormones and other proteinaceous and nonproteinaceous solutes.

As used herein, the terms “polypeptide,” “peptide” and “protein” are used interchangeably herein to mean a polymer comprising two or more amino acids joined to each other by peptide bonds or modified peptide bonds, i.e., peptide isosteres. Polypeptide refers to both short chains, commonly referred to as peptides, glycopeptides or oligomers, and to longer chains, generally referred to as proteins. Polypeptides may contain amino acids other than the 20 gene-encoded amino acids. Polypeptides include amino acid sequences modified either by natural processes, such as post-translational processing, or by chemical modification techniques that are well known in the art.

As used herein, the term “simultaneous” therapeutic use refers to the administration of at least two active ingredients by the same route and at the same time or at substantially the same time.

As used herein, the term “separate” therapeutic use refers to an administration of at least two active ingredients at the same time or at substantially the same time by different routes.

As used herein, the term “sequential” therapeutic use refers to administration of at least two active ingredients at different times, the administration route being identical or different. More particularly, sequential use refers to the whole administration of one of the active ingredients before administration of the other or others commences. It is thus possible to administer one of the active ingredients over several minutes, hours, or days before administering the other active ingredient or ingredients. There is no simultaneous treatment in this case.

As used herein, the terms “treating” or “treatment” or “alleviation” refers to both therapeutic treatment and prophylactic measures, wherein the object is to treat or slow down (lessen) the targeted pathologic condition or disorder. A subject is successfully “treated” for cognitive dysfunction if, after receiving a therapeutic amount of the aromatic-cationic peptides according to the methods described herein, the subject shows observable and/or measurable reduction in or absence of one or more signs and symptoms of cognitive dysfunction. It is also to be appreciated that the various modes of treatment or prevention of medical conditions as described are intended to mean “substantial”, which includes total but also less than total treatment or prevention, and wherein some biologically or medically relevant result is achieved.

As used herein, “prevention” or “preventing” of a disorder or condition refers to a compound that, in a statistical sample, reduces the occurrence of the disorder or condition in the treated sample relative to an untreated control sample, or delays the onset or reduces the severity of one or more symptoms of the disorder or condition relative to the untreated control sample.

### Aromatic-Cationic Peptides

The present technology relates to the treatment or prevention of cognitive dysfunction by administration of aromatic-cationic peptides of the present technology. It is expected that administration of aromatic-cationic peptides will not only be effective for the treatment or prevention of cognitive dysfunction, but that administration of the peptides in combination with additional therapeutic agents will have synergistic effects in the treatment or prevention of the disease or condition. By way of example only, but not by way of limitation, in some embodiments, aromatic-cationic peptides are administered in combination with conventional or newly developed agents, e.g., for the treatment of mitochondrial encephalopathy, thereby treating, preventing or ameliorating cognitive dysfunction and the underlying cause (e.g., the disease).

The aromatic-cationic peptides are water-soluble and highly polar. Despite these properties, the peptides can readily penetrate cell membranes. The aromatic-cationic peptides typically include a minimum of three amino acids or a minimum of four amino acids, covalently joined by peptide bonds. The maximum number of amino acids present in the aromatic-cationic peptides is about twenty amino acids covalently joined by peptide bonds. Suitably, the maximum number of amino acids is about twelve, about nine, or about six.

The amino acids of the aromatic-cationic peptides can be any amino acid. As used herein, the term “amino acid” is used to refer to any organic molecule that contains at least one amino group and at least one carboxyl group. Typically, at least one amino group is at the α position relative to a carboxyl group. The amino acids may be naturally occurring. Naturally occurring amino acids include, for example, the twenty most common levorotatory (L) amino acids normally found in mammalian proteins, i.e., alanine (Ala), arginine (Arg), asparagine (Asn), aspartic acid (Asp), cysteine (Cys), glutamine (Gin), glutamic acid (Glu), glycine (Gly), histidine (His), isoleucine (Ile), leucine (Leu), lysine (Lys), methionine (Met), phenylalanine (Phe), proline (Pro), serine (Ser), threonine (Thr), tryptophan, (Trp), tyrosine (Tyr), and valine (Val). Other naturally occurring amino acids include, for example, amino acids that are synthesized in metabolic processes not associated with protein synthesis. For example, the amino acids ornithine and citrulline are synthesized in mammalian metabolism during the production of urea. Another example of a naturally occurring amino acid include hydroxyproline (Hyp).

The peptides optionally contain one or more non-naturally occurring amino acids. Suitably, the peptide has no amino acids that are naturally occurring. The non-naturally occurring amino acids may be levorotary (L-), dextrorotatory (D-), or mixtures thereof. Non-naturally occurring amino acids are those amino acids that typically are not synthesized in normal metabolic processes in living organisms, and do not naturally occur in proteins. In addition, the non-naturally occurring amino acids suitably are also not recognized by common proteases. The non-naturally occurring amino acid can be present at any position in the peptide. For example, the non-naturally occurring amino acid can be at the N-terminus, the C-terminus, or at any position between the N-terminus and the C-terminus.

The non-natural amino acids may, for example, comprise alkyl, aryl, or alkylaryl groups not found in natural amino acids. Some examples of non-natural alkyl amino acids include α-aminobutyric acid, β-aminobutyric acid, γ-aminobutyric acid, δ-aminovaleric acid, and ε-aminocaproic acid. Some examples of non-natural aryl amino acids include ortho-, meta, and para-aminobenzoic acid. Some examples of non-natural alkylaryl amino acids include ortho-, meta-, and para-aminophenylacetic acid, and γ-phenyl-β-aminobutyric acid. Non-naturally occurring amino acids include derivatives of naturally occurring amino acids. The derivatives of naturally occurring amino acids may, for example, include the addition of one or more chemical groups to the naturally occurring amino acid.

For example, one or more chemical groups can be added to one or more of the 2′, 3′, 4′, 5′, or 6′ position of the aromatic ring of a phenylalanine or tyrosine residue, or the 4′, 5′, 6′, or 7′ position of the benzo ring of a tryptophan residue. The group can be any chemical group that can be added to an aromatic ring. Some examples of such groups include branched or unbranched C₁-C₄ alkyl, such as methyl, ethyl, n-propyl, isopropyl, butyl, isobutyl, or t-butyl, C₁-C₄ alkyloxy (i.e., alkoxy), amino, C₁-C₄ alkylamino and C₁-C₄ dialkylamino (e.g., methylamino, dimethylamino), nitro, hydroxyl, halo (i.e., fluoro, chloro, bromo, or iodo). Some specific examples of non-naturally occurring derivatives of naturally occurring amino acids include norvaline (Nva) and norleucine (Nle).

Another example of a modification of an amino acid in a peptide is the derivatization of a carboxyl group of an aspartic acid or a glutamic acid residue of the peptide. One example of derivatization is amidation with ammonia or with a primary or secondary amine, e.g. methylamine, ethylamine, dimethylamine or diethylamine. Another example of derivatization includes esterification with, for example, methyl or ethyl alcohol. Another such modification includes derivatization of an amino group of a lysine, arginine, or histidine residue. For example, such amino groups can be acylated. Some suitable acyl groups include, for example, a benzoyl group or an alkanoyl group comprising any of the C₁-C₄ alkyl groups mentioned above, such as an acetyl or propionyl group.

The non-naturally occurring amino acids may be resistant or insensitive to common proteases. Examples of non-naturally occurring amino acids that are resistant or insensitive to proteases include the dextrorotatory (D-) form of any of the above-mentioned naturally occurring L-amino acids, as well as L- and/or D-non-naturally occurring amino acids. The D-amino acids do not normally occur in proteins, although they are found in certain peptide antibiotics that are synthesized by means other than the normal ribosomal protein synthetic machinery of the cell. As used herein, the D-amino acids are considered to be non-naturally occurring amino acids.

In order to minimize protease sensitivity, the peptides should have less than five, less than four, less than three, or less than two contiguous L-amino acids recognized by common proteases, irrespective of whether the amino acids are naturally or non-naturally occurring. Suitably, the peptide has only D-amino acids, and no L-amino acids. If the peptide contains protease sensitive sequences of amino acids, at least one of the amino acids is a non-naturally-occurring D-amino acid, thereby conferring protease resistance. An example of a protease sensitive sequence includes two or more contiguous basic amino acids that are readily cleaved by common proteases, such as endopeptidases and trypsin. Examples of basic amino acids include arginine, lysine and histidine.

The aromatic-cationic peptides should have a minimum number of net positive charges at physiological pH in comparison to the total number of amino acid residues in the peptide. The minimum number of net positive charges at physiological pH will be referred to below as (pₘ). The total number of amino acid residues in the peptide will be referred to below as (r). The minimum number of net positive charges discussed below is all at physiological pH. The term “physiological pH” as used herein refers to the normal pH in the cells of the tissues and organs of the mammalian body. For instance, the physiological pH of a human is normally approximately 7.4, but normal physiological pH in mammals may be any pH from about 7.0 to about 7.8.

“Net charge” as used herein refers to the balance of the number of positive charges and the number of negative charges carried by the amino acids present in the peptide. In this specification, it is understood that net charges are measured at physiological pH. The naturally occurring amino acids that are positively charged at physiological pH include L-lysine, L-arginine, and L-histidine. The naturally occurring amino acids that are negatively charged at physiological pH include L-aspartic acid and L-glutamic acid.

Typically, a peptide has a positively charged N-terminal amino group and a negatively charged C-terminal carboxyl group. The charges cancel each other out at physiological pH. As an example of calculating net charge, the peptide Tyr-Arg-Phe-Lys-Glu-His-Trp-D-Arg has one negatively charged amino acid (i.e., Glu) and four positively charged amino acids (i.e., two Arg residues, one Lys, and one His). Therefore, the above peptide has a net positive charge of three.

In one embodiment, the aromatic-cationic peptides have a relationship between the minimum number of net positive charges at physiological pH (pₘ) and the total number of amino acid residues (r) wherein 3pₘ is the largest number that is less than or equal to r+1. In this embodiment, the relationship between the minimum number of net positive charges (pₘ) and the total number of amino acid residues (r) is as follows:

TABLE 1
Amino acid number and net positive charges (3pₘ ≦ p + 1)

| (r) | 3 | 4 | 5 | 6 | 7 | 8 | 9 | 10 | 11 | 12 | 13 | 14 | 15 | 16 | 17 | 18 | 19 | 20 |
|---|---|---|---|---|---|---|---|---|---|---|---|---|---|---|---|---|---|---|
| (pₘ) | 1 | 1 | 2 | 2 | 2 | 3 | 3 | 3 | 4 | 4 | 4 | 5 | 5 | 5 | 6 | 6 | 6 | 7 |

In another embodiment, the aromatic-cationic peptides have a relationship between the minimum number of net positive charges (pₘ) and the total number of amino acid residues (r) wherein 2pₘ is the largest number that is less than or equal to r+1. In this embodiment, the relationship between the minimum number of net positive charges (pₘ) and the total number of amino acid residues (r) is as follows:

TABLE 2
Amino acid number and net positive charges (2pₘ ≦ p + 1)

| (r) | 3 | 4 | 5 | 6 | 7 | 8 | 9 | 10 | 11 | 12 | 13 | 14 | 15 | 16 | 17 | 18 | 19 | 20 |
|---|---|---|---|---|---|---|---|---|---|---|---|---|---|---|---|---|---|---|
| (pₘ) | 2 | 2 | 3 | 3 | 4 | 4 | 5 | 5 | 6 | 6 | 7 | 7 | 8 | 8 | 9 | 9 | 10 | 10 |

In one embodiment, the minimum number of net positive charges (pₘ) and the total number of amino acid residues (r) are equal. In another embodiment, the peptides have three or four amino acid residues and a minimum of one net positive charge, a minimum of two net positive charges, or a minimum of three net positive charges.

It is also important that the aromatic-cationic peptides have a minimum number of aromatic groups in comparison to the total number of net positive charges (pₜ). The minimum number of aromatic groups will be referred to below as (a). Naturally occurring amino acids that have an aromatic group include the amino acids histidine, tryptophan, tyrosine, and phenylalanine. For example, the hexapeptide Lys-Gln-Tyr-D-Arg-Phe-Trp has a net positive charge of two (contributed by the lysine and arginine residues) and three aromatic groups (contributed by tyrosine, phenylalanine and tryptophan residues).

The aromatic-cationic peptides should also have a relationship between the minimum number of aromatic groups (a) and the total number of net positive charges at physiological pH (pₜ) wherein 3a is the largest number that is less than or equal to pₜ+1, except that when pₜ is 1, a may also be 1. In this embodiment, the relationship between the minimum number of aromatic groups (a) and the total number of net positive charges (pₜ) is as follows:

TABLE 3
Aromatic groups and net positive charges (3a ≦ pₜ + 1 or a = pₜ = 1)

| (pₜ) | 1 | 2 | 3 | 4 | 5 | 6 | 7 | 8 | 9 | 10 | 11 | 12 | 13 | 14 | 15 | 16 | 17 | 18 | 19 | 20 |
|---|---|---|---|---|---|---|---|---|---|---|---|---|---|---|---|---|---|---|---|---|
| (a) | 1 | 1 | 1 | 1 | 2 | 2 | 2 | 3 | 3 | 3 | 4 | 4 | 4 | 5 | 5 | 5 | 6 | 6 | 6 | 7 |

In another embodiment, the aromatic-cationic peptides have a relationship between the minimum number of aromatic groups (a) and the total number of net positive charges (pₜ) wherein 2a is the largest number that is less than or equal to pₜ+1. In this embodiment, the relationship between the minimum number of aromatic amino acid residues (a) and the total number of net positive charges (pₜ) is as follows:

TABLE 4
Aromatic groups and net positive charges (2a ≦ pₜ + 1 or a = pₜ = 1)

| (pₜ) | 1 | 2 | 3 | 4 | 5 | 6 | 7 | 8 | 9 | 10 | 11 | 12 | 13 | 14 | 15 | 16 | 17 | 18 | 19 | 20 |
|---|---|---|---|---|---|---|---|---|---|---|---|---|---|---|---|---|---|---|---|---|
| (a) | 1 | 1 | 2 | 2 | 3 | 3 | 4 | 4 | 5 | 5 | 6 | 6 | 7 | 7 | 8 | 8 | 9 | 9 | 10 | 10 |

In another embodiment, the number of aromatic groups (a) and the total number of net positive charges (pₜ) are equal.

Carboxyl groups, especially the terminal carboxyl group of a C-terminal amino acid, may be amidated with, for example, ammonia to form the C-terminal amide. Alternatively, the terminal carboxyl group of the C-terminal amino acid may be amidated with any primary or secondary amine. The primary or secondary amine may, for example, be an alkyl, especially a branched or unbranched C₁-C₄ alkyl, or an aryl amine. Accordingly, the amino acid at the C-terminus of the peptide may be converted to an amido, N-methylamido, N-ethylamido, N,N-dimethylamido, N,N-diethylamido, N-methyl-N-ethylamido, N-phenylamido or N-phenyl-N-ethylamido group. The free carboxylate groups of the asparagine, glutamine, aspartic acid, and glutamic acid residues not occurring at the C-terminus of the aromatic-cationic peptides may also be amidated wherever they occur within the peptide. The amidation at these internal positions may be with ammonia or any of the primary or secondary amines described above.

In one embodiment, the aromatic-cationic peptide is a tripeptide having two net positive charges and at least one aromatic amino acid. In a particular embodiment, the aromatic-cationic peptide is a tripeptide having two net positive charges and two aromatic amino acids.

Aromatic-cationic peptides include, but are not limited to, the following peptide examples:

Lys-D-Arg-Tyr-NH₂
Phe-D-Arg-His
D-Tyr-Trp-Lys-NH₂
Trp-D-Lys-Tyr-Arg-NH₂
Tyr-His-D-Gly-Met
Phe-Arg-D-His-Asp
Tyr-D-Arg-Phe-Lys-Glu-NH₂
Met-Tyr-D-Lys-Phe-Arg
D-His-Glu-Lys-Tyr-D-Phe-Arg
Lys-D-Gln-Tyr-Arg-D-Phe-Trp-NH₂
Phe-D-Arg-Lys-Trp-Tyr-D-Arg-His
Gly-D-Phe-Lys-Tyr-His-D-Arg-Tyr-NH₂
Val-D-Lys-His-Tyr-D-Phe-Ser-Tyr-Arg-NH₂
Trp-Lys-Phe-D-Asp-Arg-Tyr-D-His-Lys
Lys-Trp-D-Tyr-Arg-Asn-Phe-Tyr-D-His-NH₂
Thr-Gly-Tyr-Arg-D-His-Phe-Trp-D-His-Lys
Asp-D-Trp-Lys-Tyr-D-His-Phe-Arg-D-Gly-Lys-NH₂
D-His-Lys-Tyr- D-Phe-Glu- D-Asp- D-His- D-Lys-Arg-
Trp-NH₂
Ala-D-Phe-D-Arg-Tyr-Lys-D-Trp-His-D-Tyr-Gly-Phe
Tyr-D-His-Phe- D-Arg-Asp-Lys- D-Arg-His-Trp-D-His-
Phe
Phe-Phe-D-Tyr-Arg-Glu-Asp-D-Lys-Arg-D-Arg-His-Phe-
NH₂
Phe-Try-Lys-D-Arg-Trp-His-D-Lys-D-Lys-Glu-Arg-D-
Tyr-Thr
Tyr-Asp-D-Lys-Tyr-Phe- D-Lys- D-Arg-Phe-Pro-D-Tyr-
His-Lys
Glu-Arg-D-Lys-Tyr- D-Val-Phe- D-His-Trp-Arg-D-Gly-
Tyr-Arg-D-Met-NH₂
Arg-D-Leu-D-Tyr-Phe-Lys-Glu- D-Lys-Arg-D-Trp-Lys-
D-Phe-Tyr-D-Arg-Gly
D-Glu-Asp-Lys-D-Arg-D-His-Phe-Phe-D-Val-Tyr-Arg-
Tyr-D-Tyr-Arg-His-Phe-NH₂
Asp-Arg-D-Phe-Cys-Phe-D-Arg-D-Lys-Tyr-Arg-D-Tyr-
Trp-D-His-Tyr-D-Phe-Lys-Phe
His-Tyr-D-Arg-Trp-Lys-Phe-D-Asp-Ala-Arg-Cys-D-Tyr-
His-Phe-D-Lys-Tyr-His-Ser-NH₂
Gly-Ala-Lys-Phe-D-Lys-Glu-Arg-Tyr-His-D-Arg-D-Arg-
Asp-Tyr-Trp-D-His-Trp-His-D-Lys-Asp
Thr-Tyr-Arg-D-Lys-Trp-Tyr-Glu-Asp-D-Lys-D-Arg-His-
Phe-D-Tyr-Gly-Val-Ile-D-His-Arg-Tyr-Lys-NH₂

In one embodiment, a peptide that has mu-opioid receptor agonist activity has the formula Tyr-D-Arg-Phe-Lys-NH₂. Tyr-D-Arg-Phe-Lys-NH₂ has a net positive charge of three, contributed by the amino acids tyrosine, arginine, and lysine and has two aromatic groups contributed by the amino acids phenylalanine and tyrosine. The tyrosine of Tyr-D-Arg-Phe-Lys-NH₂ can be a modified derivative of tyrosine such as in 2′,6′-dimethyltyrosine (2′,6′-Dmt) to produce the compound having the formula 2′,6′-Dmt-D-Arg-Phe-Lys-NH₂. 2′,6′-Dmt-D-Arg-Phe-Lys-NH₂ has a molecular weight of 640 and carries a net three positive charge at physiological pH. 2′,6′-Dmt-D-Arg-Phe-Lys-NH₂ readily penetrates the plasma membrane of several mammalian cell types in an energy-independent manner (Zhao et al., J. Pharmacol Exp Ther. 304: 425-432, 2003).

Peptides that do not have mu-opioid receptor agonist activity generally do not have a tyrosine residue or a derivative of tyrosine at the N-terminus (i.e., amino acid position 1). The amino acid at the N-terminus can be any naturally occurring or non-naturally occurring amino acid other than tyrosine. In one embodiment, the amino acid at the N-terminus is phenylalanine or its derivative. Exemplary derivatives of phenylalanine include 2′-methylphenylalanine (Mmp), 2′,6′-dimethylphenylalanine (2′,6′-Dmp), N,2′,6′-trimethylphenylalanine (Tmp), and 2′-hydroxy-6′-methylphenylalanine (Hmp).

An example of an aromatic-cationic peptide that does not have mu-opioid receptor agonist activity has the formula Phe-D-Arg-Phe-Lys-NH₂. Alternatively, the N-terminal phenylalanine can be a derivative of phenylalanine such as 2′,6′-dimethylphenylalanine (2′,6′-Dmp). A variant of Phe-D-Arg-Phe-Lys-NH₂ containing 2′,6′-dimethylphenylalanine at amino acid position 1 has the formula 2′,6′-Dmp-D-Arg-Phe-Lys-NH₂. In one embodiment, the amino acid sequence of 2′,6′-Dmt-D-Arg-Phe-Lys-NH₂ is rearranged such that Dmt is not at the N-terminus. An example of such an aromatic-cationic peptide that does not have mu-opioid receptor agonist activity has the formula D-Arg-2′,6′-Dmt-Lys-Phe-NH₂.

Aromatic-cationic peptides and their derivatives can further include functional analogs. A peptide is considered a functional analog of if the analog has the same function as the aromatic-cationic peptide. The analog may, for example, be a substitution variant D-Arg-2′,6′-Dmt-Lys-Phe-NH₂, wherein one or more amino acids are substituted by another amino acid.

Suitable substitution variants of aromatic-cationic peptides include conservative amino acid substitutions. Amino acids may be grouped according to their physicochemical characteristics as follows:

(a) Non-polar amino acids: Ala(A) Ser(S) Thr(T) Pro(P) Gly(G) Cys (C);

(b) Acidic amino acids: Asn(N) Asp(D) Glu(E) Gln(Q);

(c) Basic amino acids: His(H) Arg(R) Lys(K);

(d) Hydrophobic amino acids: Met(M) Leu(L) Ile(I) Val(V); and

(e) Aromatic amino acids: Phe(F) Tyr(Y) Trp(W) His (H).

Substitutions of an amino acid in a peptide by another amino acid in the same group is referred to as a conservative substitution and may preserve the physicochemical characteristics of the original peptide. In contrast, substitutions of an amino acid in a peptide by another amino acid in a different group is generally more likely to alter the characteristics of the original peptide.

In some embodiments, the aromatic-cationic peptide has a formula as shown in Table 5.

TABLE 5
Peptide Analogs with Mu-Opioid Activity

| Amino Acid | Amino Acid | Amino Acid | Amino Acid | C-Terminal |
|---|---|---|---|---|
| Position 1 | Position 2 | Position 3 | Position 4 | Modification |
| Tyr | D-Arg | Phe | Lys | NH₂ |
| Tyr | D-Arg | Phe | Orn | NH₂ |
| Tyr | D-Arg | Phe | Dab | NH₂ |
| Tyr | D-Arg | Phe | Dap | NH₂ |
| 2′,6′-Dmt | D-Arg | Phe | Lys | NH₂ |
| 2′,6′-Dmt | D-Arg | Phe | Lys-NH(CH₂)₂—NH-dns | NH₂ |
| 2′,6′-Dmt | D-Arg | Phe | Lys-NH(CH₂)₂—NH-atn | NH₂ |
| 2′,6′-Dmt | D-Arg | Phe | dnsLys | NH₂ |
| 2′,6′-Dmt | D-Cit | Phe | Lys | NH₂ |
| 2′,6′-Dmt | D-Cit | Phe | Ahp | NH₂ |
| 2′,6′-Dmt | D-Arg | Phe | Orn | NH₂ |
| 2′,6′-Dmt | D-Arg | Phe | Dab | NH₂ |
| 2′,6′-Dmt | D-Arg | Phe | Dap | NH₂ |
| 2′,6′-Dmt | D-Arg | Phe | Ahp(2-aminoheptanoic acid) | NH₂ |
| Bio-2′,6′-Dmt | D-Arg | Phe | Lys | NH₂ |
| 3′,5′-Dmt | D-Arg | Phe | Lys | NH₂ |
| 3′,5′-Dmt | D-Arg | Phe | Orn | NH₂ |
| 3′,5′-Dmt | D-Arg | Phe | Dab | NH₂ |
| 3′,5′-Dmt | D-Arg | Phe | Dap | NH₂ |
| Tyr | D-Arg | Tyr | Lys | NH₂ |
| Tyr | D-Arg | Tyr | Orn | NH₂ |
| Tyr | D-Arg | Tyr | Dab | NH₂ |
| Tyr | D-Arg | Tyr | Dap | NH₂ |
| 2′,6′-Dmt | D-Arg | Tyr | Lys | NH₂ |
| 2′,6′-Dmt | D-Arg | Tyr | Orn | NH₂ |
| 2′,6′-Dmt | D-Arg | Tyr | Dab | NH₂ |
| 2′,6′-Dmt | D-Arg | Tyr | Dap | NH₂ |
| 2′,6′-Dmt | D-Arg | 2′,6′-Dmt | Lys | NH₂ |
| 2′,6′-Dmt | D-Arg | 2′,6′-Dmt | Orn | NH₂ |
| 2′,6′-Dmt | D-Arg | 2′,6′-Dmt | Dab | NH₂ |
| 2′,6′-Dmt | D-Arg | 2′,6′-Dmt | Dap | NH₂ |
| 3′,5′-Dmt | D-Arg | 3′,5′-Dmt | Arg | NH₂ |
| 3′,5′-Dmt | D-Arg | 3′,5′-Dmt | Lys | NH₂ |
| 3′,5′-Dmt | D-Arg | 3′,5′-Dmt | Orn | NH₂ |
| 3′,5′-Dmt | D-Arg | 3′,5′-Dmt | Dab | NH₂ |
| Tyr | D-Lys | Phe | Dap | NH₂ |
| Tyr | D-Lys | Phe | Arg | NH₂ |
| Tyr | D-Lys | Phe | Lys | NH₂ |
| Tyr | D-Lys | Phe | Orn | NH₂ |
| 2′,6′-Dmt | D-Lys | Phe | Dab | NH₂ |
| 2′,6′-Dmt | D-Lys | Phe | Dap | NH₂ |
| 2′,6′-Dmt | D-Lys | Phe | Arg | NH₂ |
| 2′,6′-Dmt | D-Lys | Phe | Lys | NH₂ |
| 3′,5′-Dmt | D-Lys | Phe | Orn | NH₂ |
| 3′,5′-Dmt | D-Lys | Phe | Dab | NH₂ |
| 3′,5′-Dmt | D-Lys | Phe | Dap | NH₂ |
| 3′,5′-Dmt | D-Lys | Phe | Arg | NH₂ |
| Tyr | D-Lys | Tyr | Lys | NH₂ |
| Tyr | D-Lys | Tyr | Orn | NH₂ |
| Tyr | D-Lys | Tyr | Dab | NH₂ |
| Tyr | D-Lys | Tyr | Dap | NH₂ |
| 2′,6′-Dmt | D-Lys | Tyr | Lys | NH₂ |
| 2′,6′-Dmt | D-Lys | Tyr | Orn | NH₂ |
| 2′,6′-Dmt | D-Lys | Tyr | Dab | NH₂ |
| 2′,6′-Dmt | D-Lys | Tyr | Dap | NH₂ |
| 2′,6′-Dmt | D-Lys | 2′,6′-Dmt | Lys | NH₂ |
| 2′,6′-Dmt | D-Lys | 2′,6′-Dmt | Orn | NH₂ |
| 2′,6′-Dmt | D-Lys | 2′,6′-Dmt | Dab | NH₂ |
| 2′,6′-Dmt | D-Lys | 2′,6′-Dmt | Dap | NH₂ |
| 2′,6′-Dmt | D-Arg | Phe | dnsDap | NH₂ |
| 2′,6′-Dmt | D-Arg | Phe | atnDap | NH₂ |
| 3′,5′-Dmt | D-Lys | 3′,5′-Dmt | Lys | NH₂ |
| 3′,5′-Dmt | D-Lys | 3′,5′-Dmt | Orn | NH₂ |
| 3′,5′-Dmt | D-Lys | 3′,5′-Dmt | Dab | NH₂ |
| 3′,5′-Dmt | D-Lys | 3′,5′-Dmt | Dap | NH₂ |
| Tyr | D-Lys | Phe | Arg | NH₂ |
| Tyr | D-Orn | Phe | Arg | NH₂ |
| Tyr | D-Dab | Phe | Arg | NH₂ |
| Tyr | D-Dap | Phe | Arg | NH₂ |
| 2′,6′-Dmt | D-Arg | Phe | Arg | NH₂ |
| 2′,6′-Dmt | D-Lys | Phe | Arg | NH₂ |
| 2′,6′-Dmt | D-Orn | Phe | Arg | NH₂ |
| 2′,6′-Dmt | D-Dab | Phe | Arg | NH₂ |
| 3′,5′-Dmt | D-Dap | Phe | Arg | NH₂ |
| 3′,5′-Dmt | D-Arg | Phe | Arg | NH₂ |
| 3′,5′-Dmt | D-Lys | Phe | Arg | NH₂ |
| 3′,5′-Dmt | D-Orn | Phe | Arg | NH₂ |
| Tyr | D-Lys | Tyr | Arg | NH₂ |
| Tyr | D-Orn | Tyr | Arg | NH₂ |
| Tyr | D-Dab | Tyr | Arg | NH₂ |
| Tyr | D-Dap | Tyr | Arg | NH₂ |
| 2′,6′-Dmt | D-Arg | 2′,6′-Dmt | Arg | NH₂ |
| 2′,6′-Dmt | D-Lys | 2′,6′-Dmt | Arg | NH₂ |
| 2′,6′-Dmt | D-Orn | 2′,6′-Dmt | Arg | NH₂ |
| 2′,6′-Dmt | D-Dab | 2′,6′-Dmt | Arg | NH₂ |
| 3′,5′-Dmt | D-Dap | 3′,5′-Dmt | Arg | NH₂ |
| 3′,5′-Dmt | D-Arg | 3′,5′-Dmt | Arg | NH₂ |
| 3′,5′-Dmt | D-Lys | 3′,5′-Dmt | Arg | NH₂ |
| 3′,5′-Dmt | D-Orn | 3′,5′-Dmt | Arg | NH₂ |
| Mmt | D-Arg | Phe | Lys | NH₂ |
| Mmt | D-Arg | Phe | Orn | NH₂ |
| Mmt | D-Arg | Phe | Dab | NH₂ |
| Mmt | D-Arg | Phe | Dap | NH₂ |
| Tmt | D-Arg | Phe | Lys | NH₂ |
| Tmt | D-Arg | Phe | Orn | NH₂ |
| Tmt | D-Arg | Phe | Dab | NH₂ |
| Tmt | D-Arg | Phe | Dap | NH₂ |
| Hmt | D-Arg | Phe | Lys | NH₂ |
| Hmt | D-Arg | Phe | Orn | NH₂ |
| Hmt | D-Arg | Phe | Dab | NH₂ |
| Hmt | D-Arg | Phe | Dap | NH₂ |
| Mmt | D-Lys | Phe | Lys | NH₂ |
| Mmt | D-Lys | Phe | Orn | NH₂ |
| Mmt | D-Lys | Phe | Dab | NH₂ |
| Mmt | D-Lys | Phe | Dap | NH₂ |
| Mmt | D-Lys | Phe | Arg | NH₂ |
| Tmt | D-Lys | Phe | Lys | NH₂ |
| Tmt | D-Lys | Phe | Orn | NH₂ |
| Tmt | D-Lys | Phe | Dab | NH₂ |
| Tmt | D-Lys | Phe | Dap | NH₂ |
| Tmt | D-Lys | Phe | Arg | NH₂ |
| Hmt | D-Lys | Phe | Lys | NH₂ |
| Hmt | D-Lys | Phe | Orn | NH₂ |
| Hmt | D-Lys | Phe | Dab | NH₂ |
| Hmt | D-Lys | Phe | Dap | NH₂ |
| Hmt | D-Lys | Phe | Arg | NH₂ |
| Mmt | D-Lys | Phe | Arg | NH₂ |
| Mmt | D-Orn | Phe | Arg | NH₂ |
| Mmt | D-Dab | Phe | Arg | NH₂ |
| Mmt | D-Dap | Phe | Arg | NH₂ |
| Mmt | D-Arg | Phe | Arg | NH₂ |
| Tmt | D-Lys | Phe | Arg | NH₂ |
| Tmt | D-Orn | Phe | Arg | NH₂ |
| Tmt | D-Dab | Phe | Arg | NH₂ |
| Tmt | D-Dap | Phe | Arg | NH₂ |
| Tmt | D-Arg | Phe | Arg | NH₂ |
| Hmt | D-Lys | Phe | Arg | NH₂ |
| Hmt | D-Orn | Phe | Arg | NH₂ |
| Hmt | D-Dab | Phe | Arg | NH₂ |
| Hmt | D-Dap | Phe | Arg | NH₂ |
| Hmt | D-Arg | Phe | Arg | NH₂ |

Dab = diaminobutyric
Dap = diaminopropionic acid
Dmt = dimethyltyrosine
Mmt = 2′-methyltyrosine
Tmt = N,2′,6′-trimethyltyrosine
Hmt = 2′-hydroxy,6′-methyltyrosine
dnsDap = β-dansyl-L-α,β-diaminopropionic acid
atnDap = β-anthraniloyl-L-α,β-diaminopropionic acid
Bio = biotin

Examples of other aromatic-cationic peptides that do not activate mu-opioid receptors include, but are not limited to, the aromatic-cationic peptides shown in Table 6.

TABLE 6
Peptide Analogs Lacking Mu-Opioid Activity

| Amino Acid | Amino Acid | Amino Acid | Amino Acid | C-Terminal |
|---|---|---|---|---|
| Position 1 | Position 2 | Position 3 | Position 4 | Modification |
| D-Arg | Dmt | Lys | Phe | NH₂ |
| D-Arg | Dmt | Phe | Lys | NH₂ |
| D-Arg | Phe | Lys | Dmt | NH₂ |
| D-Arg | Phe | Dmt | Lys | NH₂ |
| D-Arg | Lys | Dmt | Phe | NH₂ |
| D-Arg | Lys | Phe | Dmt | NH₂ |
| Phe | Lys | Dmt | D-Arg | NH₂ |
| Phe | Lys | D-Arg | Dmt | NH₂ |
| Phe | D-Arg | Phe | Lys | NH₂ |
| Phe | D-Arg | Dmt | Lys | NH₂ |
| Phe | D-Arg | Lys | Dmt | NH₂ |
| Phe | Dmt | D-Arg | Lys | NH₂ |
| Phe | Dmt | Lys | D-Arg | NH₂ |
| Lys | Phe | D-Arg | Dmt | NH₂ |
| Lys | Phe | Dmt | D-Arg | NH₂ |
| Lys | Dmt | D-Arg | Phe | NH₂ |
| Lys | Dmt | Phe | D-Arg | NH₂ |
| Lys | D-Arg | Phe | Dmt | NH₂ |
| Lys | D-Arg | Dmt | Phe | NH₂ |
| D-Arg | Dmt | D-Arg | Phe | NH₂ |
| D-Arg | Dmt | D-Arg | Dmt | NH₂ |
| D-Arg | Dmt | D-Arg | Tyr | NH₂ |
| D-Arg | Dmt | D-Arg | Trp | NH₂ |
| Trp | D-Arg | Phe | Lys | NH₂ |
| Trp | D-Arg | Tyr | Lys | NH₂ |
| Trp | D-Arg | Trp | Lys | NH₂ |
| Trp | D-Arg | Dmt | Lys | NH₂ |
| D-Arg | Trp | Lys | Phe | NH₂ |
| D-Arg | Trp | Phe | Lys | NH₂ |
| D-Arg | Trp | Lys | Dmt | NH₂ |
| D-Arg | Trp | Dmt | Lys | NH₂ |
| D-Arg | Lys | Trp | Phe | NH₂ |
| D-Arg | Lys | Trp | Dmt | NH₂ |
| Cha | D-Arg | Phe | Lys | NH₂ |
| Ala | D-Arg | Phe | Lys | NH₂ |

Cha = cyclohexyl alanine

The amino acids of the peptides shown in Table 5 and 6 may be in either the L- or the D-configuration.

The peptides may be synthesized by any of the methods well known in the art. Suitable methods for chemically synthesizing the protein include, for example, those described by Stuart and Young in Solid Phase Peptide Synthesis, Second Edition, Pierce Chemical Company (1984), and in Methods Enzymol. 289, Academic Press, Inc., New York (1997).

In some embodiments, aromatic-cationic peptides of the present technology, such as D-Arg-2′,6′-Dmt-Lys-Phe-NH₂, decrease cognitive dysfunction caused by, resulting from, or otherwise associated with cellular events that reduce the cerebral metabolic rate of oxygen (CMRO), e.g., mitochondrial encephalopathy, lactic acidosis, and stroke-like episodes (MELAS), cognitive decline secondary to aging, dementia (e.g., Lewy Body Disease), non-progressive brain injuries, and drug-induced mitochondrial side effects that result in cognitive dysfunction. In some embodiments, aromatic-cationic peptides decrease cognitive dysfunction. Additionally or alternatively, in some embodiments, aromatic-cationic peptides increase cerebral metabolic rate of oxygen.

Treatment of Cognitive Dysfunction with Aromatic-Cationic Peptides

General

Cognitive dysfunction is the loss of intellectual functions such as thinking, remembering, and reasoning of sufficient severity to interfere with daily functioning. Persons or patients with cognitive dysfunction have trouble, for example, with verbal recall, basic arithmetic, and concentration.

Cognitive dysfunction can be caused by conditions in which neuronal function has been reduced or degraded. In some embodiments, cognitive dysfunction is associated with deafferentation. In some embodiments, the deafferentation can be intrinsic (e.g., a condition or disease a subject was born with) or acquired (e.g., brain trauma or head injury after birth). Additionally or alternatively, in some embodiments, cognitive dysfunction is associated with a reduction in cerebral metabolic rate of oxygen (CMRO).

In some embodiments, a decrease in CMRO leads to characteristics seen in burst suppression. Burst-suppression is an electroencephalogram (EEG) pattern in which high-voltage activity alternates with isoelectric quiescence. Burst suppression is a characteristic of an inactive brain. An inactive brain and is commonly observed at deep levels of general anesthesia and in pathological conditions, such as, hypothermia, hypoxic-ischemia trauma/coma, and early infantile encephalopathy.

In some embodiments, at least one aromatic-cationic peptide of the present technology (e.g., D-Arg-2′,6′-Dmt-Lys-Phe-NH₂), or a pharmaceutically acceptable salt thereof, is used to treat or prevent cognitive dysfunction. Additionally, or alternatively, in some embodiments, at least one aromatic-cationic peptide (e.g., D-Arg-2′,6′-Dmt-Lys-Phe-NH₂), or a pharmaceutically acceptable salt thereof, treats or prevents cognitive dysfunction that is associated with deafferentation. Additionally, or alternatively, in some embodiments, deafferentation is reduced with peptide treatment. Additionally, or alternatively, in some embodiments, treatment with at least one aromatic-cationic peptide (e.g., D-Arg-2′,6′-Dmt-Lys-Phe-NH₂), or a pharmaceutically acceptable salt thereof, increases CMRO in a subject suffering from cognitive dysfunction.

In some embodiments, the cause of deafferentation is acquired. By way of example, but not by way of limitation, in some embodiments, acquired causes of deafferentation include, but are not limited to, anesthesia, non-progressive brain injuries, and drug-induced mitochondrial side effects.

In some embodiments, the cause of deafferentation is intrinsic to the subject. By way of example, but not by way of limitation, in some embodiments, intrinsic causes of deafferentation include, but are not limited to, inherited genetic diseases, e.g., mitochondrial diseases or disorders (e.g., MELAS and MERRF) and Lewy Body Disease.

Treating or Preventing Anesthesia Induced Cognitive Dysfunction Associated with CMRO

Long term or even permanent neuronal and neurological change can follow administration of anesthetic drugs. By way example, but not by way of limitation, postoperative cognitive dysfunction (POCD) is a clinical phenomenon characterized by cognitive deficits in subjects after anesthesia and surgery, especially in geriatric surgical subjects. POCD occurs in 25% of subjects over 60 years of age and about 33% in subjects over 80 years of age. In some embodiments, POCD patients exhibit reduced CMRO.

In some embodiments, at least one aromatic-cationic peptide (e.g., D-Arg-2′,6′-Dmt-Lys-Phe-NH₂), or a pharmaceutically acceptable salt thereof, is used to treat or prevent anesthesia induced cognitive dysfunction in a subject in need thereof. In some embodiments, at least one aromatic-cationic peptide (e.g., D-Arg-2′,6′-Dmt-Lys-Phe-NH₂), or a pharmaceutically acceptable salt thereof, is used to treat or prevent POCD. Additionally, or alternatively, in some embodiments, at least one aromatic-cationic peptide (e.g., D-Arg-2′,6′-Dmt-Lys-Phe-NH₂), or a pharmaceutically acceptable salt thereof, is used for increasing CMRO in a subject suffering POCD.

In some embodiments, at least one aromatic-cationic peptide (e.g., D-Arg-2′,6′-Dmt-Lys-Phe-NH₂), or a pharmaceutically acceptable salt thereof, is used to treat or prevent anesthesia induced cognitive dysfunction that is related to pre-existing age-independent anesthesia risks. By way of example, but not by limitation, such subjects include those with neuropsychiatric disorders diagnosed with POCD. In some embodiments, the neuropsychiatric disorder is associated with CMRO.

In some embodiments, treatment with at least one aromatic-cationic peptide (e.g., D-Arg-2′,6′-Dmt-Lys-Phe-NH2), or a pharmaceutically acceptable salt thereof, treats or prevents one or more symptoms associated with POCD. By way of example, but not by way of limitation, in some embodiments, symptoms associated with POCD include, but are not limited to, one or more of: impairment of memory, drowsiness, impairment of focus or attention, impairment of ability to plan, and delirium. In some embodiments, administration of at least one aromatic-cationic peptide treats or prevents CMRO associated with anesthesia.

Treating or Preventing Non-Progressive Brain Injuries Associated with CMRO

Non-progressive brain injuries are brain injuries that are not genetic. Examples of non-progressive brain injuries include, but are not limited to, traumatic brain injury (TBI), acquired brain injury (ABI), stroke, hypoxic-ischemia, encephalitis, and related acquired encephalopathies. In some embodiments, non-progressive brain injuries cause cognitive dysfunction associated with a decrease in CMRO.

In some embodiments, at least one aromatic-cationic peptide (e.g., D-Arg-2′,6′-Dmt-Lys-Phe-NH₂), or a pharmaceutically acceptable salt thereof, is used to treat or prevent cognitive dysfunction related to non-progressive brain injuries (e.g., TBI, ABI, stroke, hypoxic-ischemia, and encephalitis) in a subject in need thereof. Additionally or alternatively, in some embodiments, the non-progressive brain injury is associated with reduced CMRO. Additionally or alternatively, in some embodiments, at least one aromatic-cationic peptide (e.g., D-Arg-2′,6′-Dmt-Lys-Phe-NH₂), or a pharmaceutically acceptable salt thereof, increases CMRO in a subject suffering non-progressive brain injuries.

In some embodiments, treatment with at least one aromatic-cationic peptide (e.g., D-Arg-2′,6′-Dmt-Lys-Phe-NH₂), or a pharmaceutically acceptable salt thereof, treats or prevents one or more symptoms associated with cognitive dysfunctions related to non-progressive brain injuries. In some embodiments, the non-progressive brain injury is associated with CMRO.

By way of example, but not by way of limitation, in some embodiments, symptoms related to non-progressive brain injuries include, but are not limited to, lack of insight, memory problems, poor concentration, slowed response, poor planning/problem solving skills, and communication difficulties. In some embodiments, administration of at least one aromatic-cationic peptide treats or prevents CMRO associated with non-progressive brain injury.

Treating or Preventing Cognitive Dysfunction Related to Drug-Induced Mitochondrial Side Effects

Side effects of some drug treatments include cognitive dysfunction that results from drug-induced mitochondrial dysfunction. By way of example, but not by limitation, Highly Active Antiretroviral Therapy (HAART) can lead to cognitive dysfunction that is a result of drug-induced mitochondria dysfunction. J Antivir Antiretrovir, pages 1-6 (2012). HAART has significantly reduced AIDS-related morbidity and mortality. However the prevalence of HIV-1-Associated Neurocognitive Disorders (HAND) has been on the rise in the post-HAART era. A majority of the side effects of HAART can be attributed directly, or indirectly, to mitochondrial dysfunction.

Another non-limiting example of cognitive dysfunction that results from drug-induced mitochondria dysfunction is inhibition of mitochondrial support of axonal transport. Axonal transport of mitochondria is critical for proper neuronal function. Studies have shown that serotonin enhances mitochondrial movement in the axons. See, e.g., Chen et al., Mol. Cell Neurosci. 36(4): 472-483 (Aug. 15, 2007). By way of example, but not by way of limitation, dopamine can inhibit axonal transport of mitochondria. Dopamine is used to treat a variety of diseases or disorders, including, but not limited to, hypotension, bradycardia, circulatory shock, and cardiac arrest. In some embodiments, drug-induced mitochondrial side effects cause cognitive dysfunction associated with a decrease in CMRO.

In some embodiments, at least one aromatic-cationic peptide (e.g., D-Arg-2′,6′-Dmt-Lys-Phe-NH₂), or a pharmaceutically acceptable salt thereof, is used to treat or prevent cognitive dysfunction related to drug-induced mitochondrial side effects (e.g., via drug treatment of HIV) in a subject in need thereof. Additionally, or alternatively, in some embodiments, at least one aromatic-cationic peptide (e.g., D-Arg-2′,6′-Dmt-Lys-Phe-NH₂), or a pharmaceutically acceptable salt thereof, is used for increasing CMRO in a subject suffering from cognitive dysfunction related to drug-induced mitochondrial side effects.

In some embodiments, cognitive dysfunction that results from drug-induced mitochondria dysfunction is the result of inhibition of mitochondrial support of axonal transport. In some embodiments, drug-induced mitochondrial dysfunction is the result of serotonergic inhibition of mitochondrial support of axonal transport.

In some embodiments, treatment with at least one aromatic-cationic peptide (e.g., D-Arg-2′,6′-Dmt-Lys-Phe-NH₂), or a pharmaceutically acceptable salt thereof, treats or prevents one or more symptoms associated with cognitive dysfunction related to drug-induced mitochondrial side effects.

By way of example, but not by way of limitation, in some embodiments, symptoms related to cognitive dysfunction caused by drug-induced mitochondrial dysfunction such as HAND include, but are not limited to, depression, dementia, motor dysfunction, speech problems, trouble with memory, poor concentration, and behavioral change. In some embodiments, administration of at least one aromatic-cationic peptide treats or prevents CMRO associated with drug-induced mitochondrial side effects.

Treating or Preventing Cognitive and/or Neurological Dysfunctions Related to Mitochondrial Disorders

Some mitochondrial disorders have associated cognitive and/or neurological dysfunctions. By way of example, but not by limitation, mitochondrial encephalomyopathy, lactic acidosis, and stroke-like episodes (MELAS) is a family of mitochondrial cytopathies, i.e., disorders caused by dysfunctional mitochondria. Other mitochondrial cytopathies include Leber's hereditary optic neuropathy and Myoclonic Epilepsy with Ragged Red Fibers (MERRF or MERRF Syndrome). A feature of these diseases is that they are caused by inherited defects in the mitochondrial genome. In some embodiments, mitochondrial diseases or disorders (e.g., MELAS, Leber's hereditary optic neuropathy, MERRF or MERRF Syndrome) cause cognitive and/or neurological dysfunction associated with a decrease in CMRO.

In some embodiments, at least one aromatic-cationic peptide (e.g., D-Arg-2′,6′-Dmt-Lys-Phe-NH₂), or a pharmaceutically acceptable salt thereof, is used to treat or prevent cognitive and/or neurological dysfunctions related to mitochondrial diseases or disorders (e.g., MELAS and MERRF) in a subject in need thereof. Additionally, or alternatively, in some embodiments, at least one aromatic-cationic peptide (e.g., D-Arg-2′,6′-Dmt-Lys-Phe-NH₂), or a pharmaceutically acceptable salt thereof, is used for increasing CMRO in a subject suffering cognitive and/or neurological dysfunctions related to mitochondrial disorders.

Additionally, or alternatively, in some embodiments, aromatic-cationic peptides are used to treat or prevent impaired neurological brain functions that are secondary to inborn genetic errors of mitochondrial function, and in some embodiments that are associated with reduced CMRO.

In some embodiments, administration of at least one aromatic-cationic peptide (e.g., D-Arg-2′,6′-Dmt-Lys-Phe-NH₂), or a pharmaceutically acceptable salt thereof, treats or prevents one or more symptoms associated with cognitive and/or neurological dysfunctions related to mitochondrial disorders.

By way of example, but not by way of limitation, in some embodiments, cognitive and/or neurological dysfunctions associated with MELAS include, but are not limited to, seizures, ischemic strokes, altered consciousness, vision abnormalities, severe headaches, and dementia.

By way of example, but not by way of limitation, in some embodiments, cognitive and/or neurological dysfunctions associated with MERRF include, but are not limited to, muscle twitches, muscle weakness, progressive muscle stiffness, seizures, difficulty coordinating movements, a loss of sensation in the extremities, slow deterioration of intellectual function, hearing loss, and optic atrophy. In some embodiments, administration of at least one aromatic-cationic peptide treats or prevents CMRO associated with mitochondrial disorders.

Treating or Preventing Cognitive Dysfunction in Lewy Body Disease

Lewy Body Disease is one of the most common causes of dementia in the elderly. The dementia is severe enough to affect normal activities and relationships. Lewy Body Disease results when abnormal structures, called Lewy bodies, build up in areas of the brain. In some embodiments, Lewy Body Disease causes cognitive dysfunction associated with a decrease in CMRO.

In some embodiments, at least one aromatic-cationic peptide (e.g., D-Arg-2′,6′-Dmt-Lys-Phe-NH₂), or a pharmaceutically acceptable salt thereof, is used to treat or prevent cognitive dysfunction related to Lewy Body Disease in a subject in need thereof. Additionally, or alternatively, in some embodiments, at least one aromatic-cationic peptide (e.g., D-Arg-2′,6′-Dmt-Lys-Phe-NH₂), or a pharmaceutically acceptable salt thereof, is used for increasing CMRO in a subject suffering from Lewy Body Disease.

In some embodiments, administration of at least one aromatic-cationic peptide (e.g., D-Arg-2′,6′-Dmt-Lys-Phe-NH₂), or a pharmaceutically acceptable salt thereof, treats or prevents one or more symptoms associated with cognitive dysfunctions related to Lewy Body Disease. By way of example, but not by way of limitation, in some embodiments, symptoms related to Lewy Body Disease include, but are not limited to, changes in alertness and attention, hallucinations, confusion, and loss of memory. In some embodiments, administration of at least one aromatic-cationic peptide treats or prevents CMRO associated with Lewy Body Disease.

### Prophylactic and Therapeutic Uses of Aromatic-Cationic Peptides.

The aromatic-cationic peptides described herein are useful to prevent or treat disease. Specifically, the disclosure provides for both prophylactic and therapeutic methods of treating a subject at risk of (or at increased risk of) cognitive dysfunction, e.g., cognitive dysfunction associated with decreased CMRO. Accordingly, the present methods provide for the prevention and/or treatment of cognitive dysfunction in a subject by administering an effective amount of an aromatic-cationic peptide to a subject in need thereof. For example, a subject can be administered an aromatic-cationic peptide compositions in an effort to improve one or more of the factors contributing to cognitive dysfunction. Exemplary therapeutic and/or prophylactic uses of the aromatic-cationic peptides of the present disclosure (e.g., D-Arg-2′,6′-Dmt-Lys-Phe-NH₂), or pharmaceutically acceptable salts thereof, include but are not limited to one or more of the following general areas (some may overlap): (i) problems of neurological function secondary to inborn genetic errors of mitochondrial function (mitochondrial disorders such MELAS, and their neurological dysfunctions including seizures, ischemic stroke, etc.); (ii) cognitive decline secondary to aging, age related risks of post-operative cognitive disturbance with anesthesia (POCD); (iii) fluctuations of cognitive function in Lewy Body Disease which are specifically proposed to arise from energy crises in cholinergic, noradrenergic, and dopaminergic cell types affected the disorder; (iv) non-progressive brain injuries including, but not limited to, traumatic brain injury, stroke, hypoxic-ischemic injuries, anoxia, encephalitis, and related acquired encephalopathies; (v) drug-induced mitochondrial side effects specific to neuronal function (e.g. HAND and inhibition of mitochondrial axonal transport); (vi) cognitive dysfunction associated with decreased CMRO. By way of example, but not by way of limitation, in some embodiments, subjects with pre-existing risk for cognitive dysfunction following surgery (e.g. cardiac bypass subjects), or aged past 60, will benefit from pre-treatment with the aromatic-cationic peptides of the present disclosure (e.g., D-Arg-2′,6′-Dmt-Lys-Phe-NH₂), and/or short-term post anesthesia treatment. By way of example, but not by way of limitation, the present technology provides methods to treat and prevent refractory generalized seizures by administering the aromatic-cationic peptides of the present disclosure (e.g., D-Arg-2′,6′-Dmt-Lys-Phe-NH₂). In addition, the aromatic-cationic peptides of the present disclosure (e.g., D-Arg-2′,6′-Dmt-Lys-Phe-NH₂), can be used as adjuvant to limit mitochondrial side effect specific to neuronal function (e.g. serotinergic inhibition of mitochondrial support of axonal transport).

One aspect of the technology includes methods for treating the symptoms of cognitive dysfunction in a subject for therapeutic purposes. In therapeutic applications, compositions or medicaments are administered to a subject suspected of having, or already suffering from such a disease in an amount sufficient to cure, or at least partially arrest, the symptoms of the disease, including its complications and intermediate pathological phenotypes in development of the disease. As such, the disclosure provides methods of treating an individual afflicted with cognitive dysfunction.

In one aspect, the technology provides a method for preventing cognitive dysfunction (e.g., cognitive dysfunction associated with reduced CMRO) in a subject by administering to the subject an aromatic-cationic peptide that modulates one or more signs or symptoms of cognitive dysfunction. Subjects at risk for cognitive dysfunction can be identified by, e.g., any or a combination of diagnostic or prognostic assays as described herein. Any method known in the art to identify a subject at risk can be used. By way of example, but not by way of limitation, in some embodiments, methods for identify a subject at risk for cognitive dysfunction include, but are not limited to, psychological evaluations, reasoning tests, memory tests, cognitive impairment test (e.g., the Kingshill test), and mental tests (e.g., the Abbreviated Mental Test or Mini Mental State Examination (MMSE)). In some embodiments, the CMRO levels of the subject are also measured, e.g., through measuring burst suppression. In prophylactic applications, pharmaceutical compositions or medicaments of aromatic-cationic peptides are administered to a subject at increased risk of a disease or condition in an amount sufficient to eliminate or reduce the risk, lessen the severity, or delay the outset of the disease, including biochemical, histologic and/or behavioral symptoms of the disease, its complications and intermediate pathological phenotypes presenting during development of the disease. Administration of prophylactic aromatic-cationic peptides of the present technology can occur prior to the manifestation of symptoms characteristic of the aberrancy, such that a disease or disorder is prevented or, alternatively, delayed in its progression.

### Determination of the Biological Effect of the Aromatic-Cationic Peptide-Based Therapeutic

In various embodiments, suitable in vitro or in vivo assays are performed to determine the effect of a specific aromatic-cationic peptide-based therapeutic and whether its administration is indicated for treatment. In various embodiments, in vitro assays can be performed with representative cells of the type(s) involved in the subject's disorder, to determine if a given aromatic-cationic peptide-based therapeutic exerts the desired effect upon the cell type(s). Compounds for use in therapy can be tested in suitable animal model systems including, but not limited to rats, mice, chicken, cows, monkeys, rabbits, and the like, prior to testing in human subjects. Similarly, for in vivo testing, any of the animal model system known in the art can be used prior to administration to human subjects. In one embodiment, administration of an aromatic-cationic peptide to a subject exhibiting symptoms associated with cognitive dysfunction will cause an improvement in one or more of those symptoms. Any method known in the art to identify improvement of one or more symptoms of cognitive dysfunction can be used. By way of example, but not by way of limitation, in some embodiments, methods for identifying improvement in cognitive dysfunction includes, but are not limited to, psychological evaluations, reasoning tests, memory tests, cognitive impairment test (e.g., the Kingshill test), and mental tests (e.g., the Abbreviated Mental Test or Mini Mental State Examination (MMSE)). In some embodiments, the CMRO levels of the subject are also measured, e.g., through measuring burst suppression.

### Modes of Administration and Effective Dosages

Any method known to those in the art for contacting a cell, organ or tissue with a peptide may be employed. Suitable methods include in vitro, ex vivo, or in vivo methods. In vivo methods typically include the administration of an aromatic-cationic peptide, such as those described above, to a mammal, such as a human. When used in vivo for therapy, the aromatic-cationic peptides are administered to the subject in effective amounts (i.e., amounts that have desired therapeutic effect). The dose and dosage regimen will depend upon the extent or severity of cognitive dysfunction in the subject, the characteristics of the particular aromatic-cationic peptide used, e.g., its therapeutic index, the subject, and the subject's history.

The effective amount may be determined during pre-clinical trials and clinical trials by methods familiar to physicians and clinicians. An effective amount of a peptide useful in the methods of the present invention, such as in a pharmaceutical composition, may be administered to a mammal in need thereof by any of a number of well-known methods for administering pharmaceutical compounds. In some embodiments, the peptide may be administered systemically, topically, or intraocularly.

The peptide may be formulated as a pharmaceutically acceptable salt. Salts derived from pharmaceutically acceptable inorganic bases include ammonium, calcium, copper, ferric, ferrous, lithium, magnesium, manganic, manganous, potassium, sodium, and zinc salts, and the like. Salts derived from pharmaceutically acceptable organic bases include salts of primary, secondary and tertiary amines, including substituted amines, cyclic amines, naturally-occurring amines and the like, such as arginine, betaine, caffeine, choline, N,N′-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperadine, polyamine resins, procaine, purines, theobromine, triethylamine, trimethylamine, tripropylamine, tromethamine and the like. Salts derived from pharmaceutically acceptable inorganic acids include salts of boric, carbonic, hydrohalic (hydrobromic, hydrochloric, hydrofluoric or hydroiodic), nitric, phosphoric, sulfamic and sulfuric acids. Salts derived from pharmaceutically acceptable organic acids include salts of aliphatic hydroxyl acids (e.g., citric, gluconic, glycolic, lactic, lactobionic, malic, and tartaric acids), aliphatic monocarboxylic acids (e.g., acetic, butyric, formic, propionic and trifluoroacetic acids), amino acids (e.g., aspartic and glutamic acids), aromatic carboxylic acids (e.g., benzoic, p-chlorobenzoic, diphenylacetic, gentisic, hippuric, and triphenylacetic acids), aromatic hydroxyl acids (e.g., o-hydroxybenzoic, p-hydroxybenzoic, 1-hydroxynaphthalene-2-carboxylic and 3-hydroxynaphthalene-2-carboxylic acids), ascorbic, dicarboxylic acids (e.g., fumaric, maleic, oxalic and succinic acids), glucuronic, mandelic, muck, nicotinic, orotic, pamoic, pantothenic, sulfonic acids (e.g., benzenesulfonic, camphosulfonic, edisylic, ethanesulfonic, isethionic, methanesulfonic, naphthalenesulfonic, naphthalene-1,5-disulfonic, naphthalene-2,6-disulfonic and p-toluenesulfonic acids), xinafoic acid, and the like. In some embodiments, the salt is an acetate or trifluoroacetate salt.

The aromatic-cationic peptides described herein can be incorporated into pharmaceutical compositions for administration, singly or in combination, to a subject for the treatment or prevention of a disorder described herein. Such compositions typically include the active agent and a pharmaceutically acceptable carrier. As used herein the term “pharmaceutically acceptable carrier” includes saline, solvents, dispersion media, coatings, antibacterial and antifungal agents, isotonic and absorption delaying agents, and the like, compatible with pharmaceutical administration. Supplementary active compounds can also be incorporated into the compositions.

Pharmaceutical compositions are typically formulated to be compatible with its intended route of administration. Examples of routes of administration include parenteral (e.g., intravenous, intradermal, intraperitoneal or subcutaneous), oral, inhalation, transdermal (topical), intraocular, iontophoretic, and transmucosal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic. For convenience of the subject or treating physician, the dosing formulation can be provided in a kit containing all necessary equipment (e.g., vials of drug, vials of diluent, syringes and needles) for a treatment course.

Pharmaceutical compositions suitable for injectable use can include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, a composition for parenteral administration must be sterile and should be fluid to the extent that easy syringability exists. It should be stable under the conditions of manufacture and storage and must be preserved against the contaminating action of microorganisms such as bacteria and fungi.

The aromatic-cationic peptide compositions can include a carrier, which can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thiomerasol, and the like. Glutathione and other antioxidants can be included to prevent oxidation. In many cases, it may be desirable to include isotonic agents, for example, sugars, polyalcohols such as mannitol, sorbitol, or sodium chloride in the composition. Prolonged absorption of the injectable compositions can be brought about by including in the composition an agent which delays absorption, for example, aluminum monostearate or gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, typical methods of preparation include vacuum drying and freeze drying, which can yield a powder of the active ingredient plus any additional desired ingredient from a previously sterile-filtered solution thereof.

For ophthalmic applications, the therapeutic compound is formulated into solutions, suspensions, and ointments appropriate for use in the eye. For ophthalmic formulations generally, see Mitra (ed.), Ophthalmic Drug Delivery Systems, Marcel Dekker, Inc., New York, N.Y. (1993) and also Havener. W. H., Ocular Pharmacology, C. V. Mosby Co., St. Louis (1983). Ophthalmic pharmaceutical compositions may be adapted for topical administration to the eye in the form of solutions, suspensions, ointments, creams or as a solid insert. For a single dose, from between 0.1 ng to 5000 μg, 1 ng to 500 μg, or 10 ng to 100 μg of the aromatic-cationic peptides can be applied to the human eye.

The ophthalmic preparation may contain non-toxic auxiliary substances such as antibacterial components which are non-injurious in use, for example, thimerosal, benzalkonium chloride, methyl and propyl paraben, benzyldodecinium bromide, benzyl alcohol, or phenylethanol; buffering ingredients such as sodium chloride, sodium borate, sodium acetate, sodium citrate, or gluconate buffers; and other conventional ingredients such as sorbitan monolaurate, triethanolamine, polyoxyethylene sorbitan monopalmitylate, ethylenediamine tetraacetic acid, and the like.

The ophthalmic solution or suspension may be administered as often as necessary to maintain an acceptable level of the aromatic-cationic peptide in the eye. Administration to the mammalian eye may be about once or twice daily.

Oral compositions generally include an inert diluent or an edible carrier. For the purpose of oral therapeutic administration, the active compound can be incorporated with excipients and used in the form of tablets, troches, or capsules, e.g., gelatin capsules. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds can be delivered in the form of an aerosol spray from a pressurized container or dispenser which contains a suitable propellant, e.g., a gas such as carbon dioxide, or a nebulizer. Such methods include those described in U.S. Pat. No. 6,468,798.

Systemic administration of a therapeutic compound as described herein can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays. For transdermal administration, the active compounds are formulated into ointments, salves, gels, or creams as generally known in the art. In one embodiment, transdermal administration may be performed by iontophoresis.

A therapeutic protein or peptide can be formulated in a carrier system. The carrier can be a colloidal system. The colloidal system can be a liposome, a phospholipid bilayer vehicle. In one embodiment, the therapeutic peptide is encapsulated in a liposome while maintaining peptide integrity. As one skilled in the art would appreciate, there are a variety of methods to prepare liposomes. (See Lichtenberg et al., Methods Biochem. Anal., 33:337-462 (1988); Anselem et al., Liposome Technology, CRC Press (1993)). Liposomal formulations can delay clearance and increase cellular uptake (See Reddy, Ann. Pharmacother., 34 (7-8):915-923 (2000)). An active agent can also be loaded into a particle prepared from pharmaceutically acceptable ingredients including, but not limited to, soluble, insoluble, permeable, impermeable, biodegradable or gastroretentive polymers or liposomes. Such particles include, but are not limited to, nanoparticles, biodegradable nanoparticles, microparticles, biodegradable microparticles, nanospheres, biodegradable nanospheres, microspheres, biodegradable microspheres, capsules, emulsions, liposomes, micelles and viral vector systems.

The carrier can also be a polymer, e.g., a biodegradable, biocompatible polymer matrix. In one embodiment, the therapeutic peptide can be embedded in the polymer matrix, while maintaining protein integrity. The polymer may be natural, such as polypeptides, proteins or polysaccharides, or synthetic, such as poly α-hydroxy acids. Examples include carriers made of, e.g., collagen, fibronectin, elastin, cellulose acetate, cellulose nitrate, polysaccharide, fibrin, gelatin, and combinations thereof. In one embodiment, the polymer is poly-lactic acid (PLA) or copoly lactic/glycolic acid (PGLA). The polymeric matrices can be prepared and isolated in a variety of forms and sizes, including microspheres and nanospheres. Polymer formulations can lead to prolonged duration of therapeutic effect. (See Reddy, Ann. Pharmacother., 34 (7-8):915-923 (2000)). A polymer formulation for human growth hormone (hGH) has been used in clinical trials. (See Kozarich and Rich, Chemical Biology, 2:548-552 (1998)).

Examples of polymer microsphere sustained release formulations are described in PCT publication WO 99/15154 (Tracy et al.), U.S. Pat. Nos. 5,674,534 and 5,716,644 (both to Zale et al.), PCT publication WO 96/40073 (Zale et al.), and PCT publication WO 00/38651 (Shah et al.). U.S. Pat. Nos. 5,674,534 and 5,716,644 and PCT publication WO 96/40073 describe a polymeric matrix containing particles of erythropoietin that are stabilized against aggregation with a salt.

In some embodiments, the therapeutic compounds are prepared with carriers that will protect the therapeutic compounds against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylacetic acid. Such formulations can be prepared using known techniques. The materials can also be obtained commercially, e.g., from Alza Corporation and Nova Pharmaceuticals. Inc. Liposomal suspensions (including liposomes targeted to specific cells with monoclonal antibodies to cell-specific antigens) can also be used as pharmaceutically acceptable carriers. These can be prepared according to methods known to those skilled in the art, for example, as described in U.S. Pat. No. 4,522,811.

The therapeutic compounds can also be formulated to enhance intracellular delivery. For example, liposomal delivery systems are known in the art, see, e.g., Chonn and Cullis, “Recent Advances in Liposome Drug Delivery Systems,” Current Opinion in Biotechnology 6:698-708 (1995); Weiner, “Liposomes for Protein Delivery: Selecting Manufacture and Development Processes.” Immunomethods 4 (3) 201-9 (1994); and Gregoriadis, “Engineering Liposomes for Drug Delivery: Progress and Problems,” Trends Biotechnol. 13 (12):527-37 (1995). Mizguchi et al., Cancer Lett. 100:63-69 (1996).

Dosage, toxicity and therapeutic efficacy of the therapeutic agents can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit high therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies ideally within a range of circulating concentrations that include the ED50 with little or no toxicity. The dosage may vary within this range depending upon the dosage form employed and the route of administration utilized. For any compound used in the methods, the therapeutically effective dose can be estimated initially from cell culture assays. A dose can be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

Typically, an effective amount of the aromatic-cationic peptides, sufficient for achieving a therapeutic or prophylactic effect, range from about 0.000001 mg per kilogram body weight per day to about 10,000 mg per kilogram body weight per day. In some embodiments, the dosage ranges are from about 0.0001 mg per kilogram body weight per day to about 100 mg per kilogram body weight per day. For example dosages can be 1 mg/kg body weight or 10 mg/kg body weight every day, every two days or every three days or within the range of 1-10 mg/kg every week, every two weeks or every three weeks. In one embodiment, a single dosage of peptide ranges from 0.1-10.000 micrograms per kg body weight. In one embodiment, aromatic-cationic peptide concentrations in a carrier range from 0.2 to 2000 micrograms per delivered milliliter. An exemplary treatment regime entails administration once per day or once a week. Intervals can also be irregular as indicated by measuring blood levels of glucose or insulin in the subject and adjusting dosage or administration accordingly. In therapeutic applications, a relatively high dosage at relatively short intervals is sometimes required until progression of the disease is reduced or terminated, or until the subject shows partial or complete amelioration of symptoms of disease. Thereafter, the subject can be administered a prophylactic regime.

In some embodiments, a subject is administered at least one aromatic-cationic peptide at least once a week for about one week, about two weeks, about three weeks, about four weeks, about five weeks, or about six weeks. In some embodiments, a subject is administered at least one aromatic-cationic peptide at least once a week for between about 6 to 24 weeks, about 8 to 22 weeks, about 10 to 20 weeks, about 12 to 18 weeks, or about 14-16 weeks.

In some embodiments, a subject is administered at least one aromatic-cationic peptide daily for about one week, about two weeks, about three weeks, about four weeks, about five weeks, or about six weeks. In some embodiments, a subject is administered at least one aromatic-cationic peptide daily for between about 6 to 24 weeks, about 8 to 22 weeks, about 10 to 20 weeks, about 12 to 18 weeks, or about 14-16 weeks.

In some embodiments, a subject is administered at least one aromatic-cationic peptide at least once a week for the remainder of his/her life. In some embodiments, a subject is administered at least one aromatic-cationic peptide daily for the remainder of his/her life.

In some embodiments, a therapeutically effective amount of an aromatic-cationic peptide may be defined as a concentration of peptide at the target tissue of 10⁻¹¹ to 10⁶ molar, e.g., approximately 10⁻⁷ molar. This concentration may be delivered by systemic doses of 0.001 to 100 mg/kg or equivalent dose by body surface area. The schedule of doses would be optimized to maintain the therapeutic concentration at the target tissue, such as by single daily or weekly administration, but also including continuous administration (e.g., parenteral infusion or transdermal application).

In some embodiments, the dosage of the aromatic-cationic peptide is provided at a “low,” “mid,” or “high” dose level. In one embodiment, the low dose is provided from about 0.0001 to about 0.5 mg/kg/h, suitably from about 0.01 to about 0.1 mg/kg/h. In one embodiment, the mid-dose is provided from about 0.1 to about 1.0 mg/kg/h, suitably from about 0.1 to about 0.5 mg/kg/h. In one embodiment, the high dose is provided from about 0.5 to about 10 mg/kg/h, suitably from about 0.5 to about 2 mg/kg/h.

The skilled artisan will appreciate that certain factors may influence the dosage and timing required to effectively treat a subject, including but not limited to, the severity of the disease or disorder, previous treatments, the general health and/or age of the subject, and other diseases present. Moreover, treatment of a subject with a therapeutically effective amount of the therapeutic compositions described herein can include a single treatment or a series of treatments.

The skilled artisan will appreciate that certain factors may influence the dosage and timing required to effectively treat a subject, including but not limited to, the severity of the disease or disorder, previous treatments, the general health and/or age of the subject, and other diseases present. Moreover, treatment of a subject with a therapeutically effective amount of the therapeutic compositions described herein can include a single treatment or a series of treatments.

The mammal treated in accordance present methods can be any mammal, including, for example, farm animals, such as sheep, pigs, cows, and horses; pet animals, such as dogs and cats; laboratory animals, such as rats, mice and rabbits. In some embodiments, the mammal is a human.

Combination Therapy with an Aromatic-Cationic Peptide and Other Therapeutic Agents

In some embodiments, at least one of the aromatic-cationic peptides described herein (or a pharmaceutically acceptable salt, ester, amide, prodrug, or solvate) in combination with at least one additional agent. By way of example only, but not by way of limitation, if a side effects experienced by a subject administered at least one aromatic-cationic peptide described herein is inflammation, then it may be appropriate to administer an anti-inflammatory agent in combination with the aromatic-cationic peptide. By way of example, but not by way of limitation, the therapeutic effectiveness of one of the compounds described herein may be enhanced by administration of an adjuvant (i.e., by itself the adjuvant may only have minimal therapeutic benefit, but in combination with another therapeutic agent, the overall therapeutic benefit to the subject is enhanced).

By way of example, but not by way of limitation, in some embodiments, at least one aromatic-cationic peptide, or a pharmaceutically acceptable salt thereof, is used in combination with at least one additional agent that also has therapeutic benefit in the prevention or treatment of cognitive dysfunction. In some embodiments, the combination of at least one aromatic-cationic peptide and additional agent produces a synergistic therapeutic effect. As used herein, a “synergistic therapeutic effect” refers to a greater-than-additive therapeutic effect, which is produced by a combination of at least two agents, and which exceeds that which would otherwise result from the sole administration of the at least two agents. For example, lower doses of one or more agents may be used in treating a disease or disorder, resulting in increased therapeutic efficacy and/or decreased side-effects.

In some embodiments, at least one aromatic-cationic peptide is used in combination with procedures that may provide additional or synergistic benefits to the subject. By way of example, but not by way of limitation, in some embodiments, at least one aromatic-cationic peptide, or a pharmaceutically acceptable salt thereof, is used in combination with electrical stimulation or optogenetic techniques in the treatment of cognitive dysfunction. e.g., cognitive dysfunction associated CMRO, caused, for example, by brain injury (e.g., non-progressive brain injuries).

Additionally or alternatively, in some embodiments, additional agent includes, but is not limited to, cholinesterase inhibitors (e.g., donepezil hydrochloride), CoQ10, L-arginine, resveratrol, SIRT1 activators, riboflavin, nicotinamide, and antipsychotics.

In some embodiments, the additional agent includes, but is not limited to, an antioxidant or an anti-inflammatory agent. By way of example, but not by way of limitation, in some embodiments, the antioxidant includes, but is not limited to, vitamin C, vitamin E, beta-carotene and other carotenoids, coenzyme Q, 4-hydroxy-2,2,6,6-tetramethylpiperidine-N-oxyl (also known as Tempol), lutein, butylated hydroxytoluene, resveratrol, a trolox analogue (PNU-83836-E), and bilberry extract.

In some embodiments, the additional agent is pyruvate.

In any case, the multiple therapeutic agents may be administered in any order or even simultaneously. If simultaneously, the multiple therapeutic agents may be provided in a single, unified form, or in multiple forms (by way of example only, either as a single solution or as two separate solutions). One of the therapeutic agents may be given in multiple doses, or both may be given as multiple doses. If not simultaneous, the timing between the multiple doses may vary from more than zero weeks to less than about four weeks, less than about six weeks, less than about 2 months, less than about 4 months, less than about 6 months, or less than about one year. In addition, the combination methods, compositions and formulations are not to be limited to the use of only two agents. By way of example only, an aromatic-cationic peptide may be provided with at least one antioxidant and at least one negatively charged phospholipid; or an aromatic-cationic peptide may be provided with at least one antioxidant and at least one inducer of nitric oxide production; or an aromatic-cationic peptide may be provided with at least one inducer of nitric oxide productions and at least one negatively charged phospholipid; and so forth.

In some embodiments, a subject is administered multiple therapeutic agents at least once a week for the remainder of his/her life. In some embodiments, a subject is administered multiple therapeutic agents daily for the remainder of his/her life.

Further combinations that may be used to benefit an individual include using genetic testing to determine whether that individual is a carrier of a mutant gene that is known to be correlated with cognitive dysfunction. Subjects possessing cognitive dysfunction-associated mutations are expected to find therapeutic and/or prophylactic benefit in the methods described herein.

### EXAMPLES

The present invention is further illustrated by the following examples, which should not be construed as limiting in any way.

### Example 1

### Prevention of Postoperative Cognitive Dysfunction in Elderly Subjects

This example demonstrates the use of D-Arg-2′,6′-Dmt-Lys-Phe-NH₂ in the prevention of postoperative cognitive dysfunction (POCD). The example demonstrates the use of D-Arg-2′,6′-Dmt-Lys-Phe-NH₂ in preventing POCD in a human.

Eighteen human subjects, whose age will be at least 60 years old, who will be subject to cardiac surgery (e.g., CABG) and who are not diagnosed with any neurodegenerative diseases, e.g., Alzheimer's or Parkinson's disease, will be divided into three groups. The first group (Group 1) will not be treated with D-Arg-2′,6′-Dmt-Lys-Phe-NH₂ pre- or post-operation. The second group (Group 2) will be treated with a control peptide pre- and post-operation. The third group (Group 3) will be treated with an effective amount of D-Arg-2′,6′-Dmt-Lys-Phe-NH₂ pre- and post-operation.

Subjects from Groups 2 and 3 will be intravenously administered about 1 mg to 10 mg or about 0.5 mg/kg to 3.0 mg/kg of control peptide or D-Arg-2′,6′-Dmt-Lys-Phe-NH₂, respectively, 30 minutes before anesthesia is given. Groups 2 and 3 will be treated with 10 mg of control peptide or D-Arg-2′,6′-Dmt-Lys-Phe-NH₂, respectively, immediately after the surgery is complete. Subjects from Groups 2 and 3 will be intravenously administered control peptide or D-Arg-2′,6′-Dmt-Lys-Phe-NH₂, respectively, at about 1 mg to 10 mg or about 0.5 mg/kg to 3.0 mg/kg once daily for three months. After three months all three groups will be tested for symptoms of cognitive dysfunction related to POCD, e.g., impairment of memory, drowsiness, impairment of focus or attention, impairment of ability to plan, and delirium. It is anticipated that Group 3 will demonstrate a reduction in cognitive dysfunctions related to POCD as compared to Groups 1 and 2.

These results will show that aromatic-cationic peptides of the present technology, such as D-Arg-2′,6′-Dmt-Lys-Phe-NH₂ are useful for preventing the onset of and/or reducing the severity of the symptoms of postoperative cognitive dysfunction in a mammalian subject. As such, aromatic-cationic peptides of the present technology, such as D-Arg-2′,6′-Dmt-Lys-Phe-NH₂ are useful in methods for preventing postoperative cognitive dysfunction in a mammalian subject in need thereof comprising administering a therapeutically effective amount of an aromatic-cationic peptide, such as D-Arg-2′,6′-Dmt-Lys-Phe-NH₂.

### Example 2

### Treating Cognitive Dysfunction in Lewy Body Disease in a Mammalian Subject

This example demonstrates the use of D-Arg-2′,6′-Dmt-Lys-Phe-NH₂ in the treatment of cognitive dysfunction in Lewy Body Disease. The example demonstrates the use of D-Arg-2′,6′-Dmt-Lys-Phe-NH₂ in treating cognitive dysfunction in Lewy Body Disease in a human.

Eighteen human subjects diagnosed with or at risk of Lewy Body Disease will be divided into three groups. The first group (Group 1) will not be treated with any peptides. The second group (Group 2) will be treated with a control peptide. The third group (Group 3) will be treated with D-Arg-2′,6′-Dmt-Lys-Phe-NH₂. Subjects from Groups 2 and 3 will be intravenously administered about 1 mg to 10 mg or about 0.5 mg/kg to 3.0 mg/kg of control peptide or D-Arg-2′,6′-Dmt-Lys-Phe-NH₂, respectively, once daily for three months. After three months all three groups will be tested for symptoms of cognitive dysfunction related to Lewy Body Disease, for example, fluctuation in autonomic processes, visual hallucinations, sleep disturbances, drowsiness, reduced alertness, and dementia. It is anticipated that Group 3 will demonstrate a reduction in cognitive dysfunction associated with Lewy Body Disease as compared to Groups 1 and 2.

These results will show that aromatic-cationic peptides of the present technology, such as D-Arg-2′,6′-Dmt-Lys-Phe-NH₂ are useful for treating the severity of the symptoms of Lewy Body Disease in a mammalian subject. As such, aromatic-cationic peptides of the present technology, such as D-Arg-2′,6′-Dmt-Lys-Phe-NH₂, are useful in methods for treating cognitive dysfunction in a mammalian subject in need thereof.

### Example 3

### Increased Cerebral Metabolic Rate of Oxygen (CMRO) in a Burst Suppression Model

This example demonstrates the use of D-Arg-2′,6′-Dmt-Lys-Phe-NH₂ in the increase of cerebral metabolic rate of oxygen (CMRO). In particular, the example demonstrates the use of D-Arg-2′,6′-Dmt-Lys-Phe-NH₂ in increasing CMRO in a burst suppression model.

A burst suppression model, as described in Ching et al., PNAS, 109(8): 3095-3100, (2012), will be used to determine the effect of D-Arg-2′,6′-Dmt-Lys-Phe-NH₂ on CMRO. A range of 10⁻⁹ to 10⁻⁶ molar concentration of D-Arg-2′,6′-Dmt-Lys-Phe-NH₂ will be assayed and compared to untreated and control peptide assays.

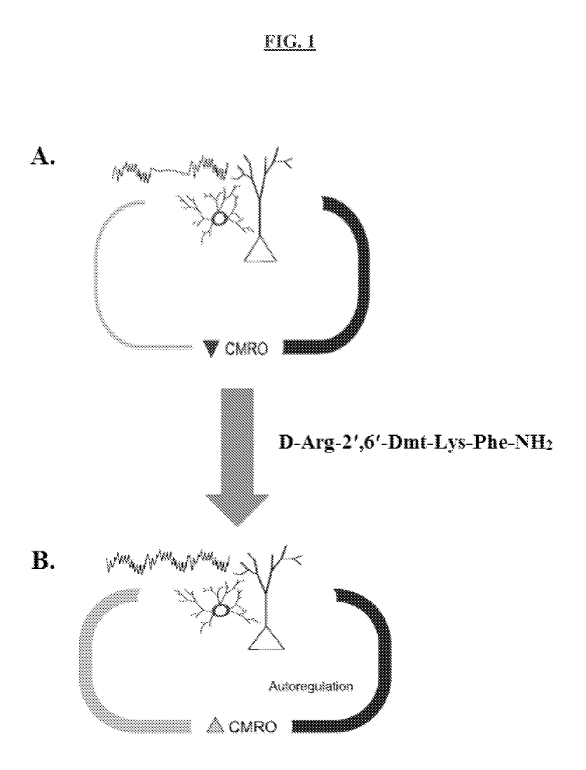

It is expected that administration of D-Arg-2′,6′-Dmt-Lys-Phe-NH₂ will lead to an increase in CMRO, wherein the higher the dose of D-Arg-2′,6′-Dmt-Lys-Phe-NH₂ the greater the increase in CMRO, as compared to untreated and control peptide assays. As seen in FIG. 1, it is expected that treatment with D-Arg-2′,6′-Dmt-Lys-Phe-NH₂ will demonstrate an increase in cerebral metabolism and/or a restoration of neuronal cellular and network function.

These results will show that aromatic-cationic peptides of the present technology, such as D-Arg-2′,6′-Dmt-Lys-Phe-NH₂ are useful in methods for increasing CMRO, wherein the methods comprise administering a therapeutically effective amount of an aromatic-cationic peptide, such as D-Arg-2′,6′-Dmt-Lys-Phe-NH₂.

### EQUIVALENTS

The present invention is not to be limited in terms of the particular embodiments described in this application, which are intended as single illustrations of individual aspects of the invention. Many modifications and variations of this invention can be made without departing from its spirit and scope, as will be apparent to those skilled in the art. Functionally equivalent methods and apparatuses within the scope of the invention, in addition to those enumerated herein, will be apparent to those skilled in the art from the foregoing descriptions. Such modifications and variations are intended to fall within the scope of the appended claims. The present invention is to be limited only by the terms of the appended claims, along with the full scope of equivalents to which such claims are entitled. It is to be understood that this invention is not limited to particular methods, reagents, compounds compositions or biological systems, which can, of course, vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

All patents, patent applications, provisional applications, and publications referred to or cited herein are incorporated by reference in their entirety, including all figures and tables, to the extent they are not inconsistent with the explicit teachings of this specification.

Other embodiments are set forth within the following claims.

1. A method for treating or preventing cognitive dysfunction associated with decreased cerebral metabolic rate of oxygen (CMRO) in a mammalian subject in need thereof, comprising administering to the subject a therapeutically effective amount of a peptide represented by the formula D-Arg-2′,6′-Dmt-Lys-Phe-NH₂ or a pharmaceutically acceptable salt thereof.

2. The method of claim 1, wherein the cognitive dysfunction is associated with deafferentation.

3. The method of claim 1, wherein the cognitive dysfunction is caused by a genetic mitochondrial disorder selected from the group consisting of mitochondrial encephalomyopathy, lactic acidosis, and stroke-like episodes (MELAS), and myoclonic epilepsy with ragged red fiber (MERRF).

4. A method for treating or preventing CMRO associated post-operative cognitive dysfunction (POCD) in a mammalian subject in need thereof, comprising administering to the subject a therapeutically effective amount of a peptide represented by the formula D-Arg-2′,6′-Dmt-Lys-Phe-NH₂ or a pharmaceutically acceptable salt thereof.

5. The method of claim 4, wherein the POCD is anesthesia induced.

6. The method of claim 1, wherein the cognitive dysfunction associated with decreased CMRO is caused by drug-induced mitochondrial dysfunction.

7. The method of claim 6, wherein the drug-induced mitochondrial dysfunction is associated with Highly Active Antiretroviral Therapy (HAART) or inhibition of mitochondrial axonal transport.

8. The method of claim 1, wherein the cognitive dysfunction associated with decreased CMRO is caused by non-progressive brain injuries, and wherein the non-progressive brain injuries is selected from encephalitis or acquired brain injury.

9. The method of claim 1, further comprising administering an additional agent.

10. The method of claim 1, wherein the subject is a human.

11. The method of claim 1, wherein the peptide is administered intraocularly, iontophoretically, orally, topically, systemically, intravenously, subcutaneously, or intramuscularly.

12. The method of claim 4, further comprising administering an additional agent.

13. The method of claim 4, wherein the subject is a human.

14. The method of claim 4, wherein the peptide is administered intraocularly, iontophoretically, orally, topically, systemically, intravenously, subcutaneously, or intramuscularly.
